(19) United States
(12) Patent Application Publication
(10) Pub. No.: US 2008/0076942 A1
(43) Pub. Date: Mar. 27, 2008

(54) AROMATIC AMINE DERIVATIVE AND ORGANIC ELECTROLUMINESCENCE DEVICE USING THE SAME

(75) Inventor: Masahiro Kawamura, Chiba (JP)

Correspondence Address: OBLON, SPIVAK, MCCLELLAND MAIER & NEUSTADT, P.C., 1940 DUKE STREET, ALEXANDRIA, VA, 22314, US

(73) Assignee: IDEMITSU KOSAN CO., LTD., Chiyoda-ku (JP)

(21) Appl. No.: 11/843,327

(22) Filed: Aug. 22, 2007

(30) Foreign Application Priority Data
Sep. 22, 2006 (JP) 2006-257387

(51) Int. Cl. H01L 51/54; C07C 211/54

(52) U.S. Cl. 564/429; 313/504; 564/434

(57) ABSTRACT

An aromatic amine derivative having a specific structure. An organic electroluminescence device which is composed of one or more organic thin film layers including at least one light emitting layer sandwiched between a cathode and an anode, wherein at least one of the organic thin film layers contains the aromatic amine derivative or a material for the organic electroluminescence device singly or as its mixture component. They provide an organic electroluminescence device with an enhanced efficiency of light emission and having prolonged lifetime emitting blue light, a novel aromatic amine derivative and a material for the organic electroluminescence device realizing the device.

### TECHNICAL FIELD

The present invention relates to an organic electroluminescence (which will be occasionally referred to as “EL”, hereinafter) device. More particularly, it relates to an organic EL device capable of being driven under a low electric voltage employing an aromatic amine derivative as a material for a hole transporting region and/or a material for a hole injecting region resultantly realizing a prolonged lifetime, an enhanced efficiency of light emission and a reasonable production cost.

### BACKGROUND ART

An organic electroluminescence (“electroluminescence” will be occasionally referred to as “EL”, hereinafter) device is a spontaneous light emitting device which utilizes the phenomenon that a fluorescent substance emits light by energy of recombination of holes injected from an anode and electrons injected from a cathode when an electric field is applied. Since an organic EL device of the laminate type driven under a low electric voltage was reported by C. W. Tang et al. of Eastman Kodak Company (C. W. Tang and S. A. Vanslyke, Applied Physics Letters, Volume 51, Page 913, 1987), many studies have been conducted on organic EL devices using organic materials as the constituting materials. Tang et al. used tris(8-quinolinolato)aluminum for the light emitting layer and a triphenyldiamine derivative for the hole transporting layer. Advantages of the laminate structure are that the efficiency of hole injection into the light emitting layer can be increased, that the efficiency of forming excitons which are formed by blocking and recombining electrons injected from the cathode can be increased, and that the excitons formed in the light emitting layer can be confined. As the structure of the organic EL device, a two-layered structure having a hole transporting (injecting) layer and an electron transportable light emitting layer and a three-layered structure having a hole transporting (injecting) layer, a light emitting layer and an electron transporting (injecting) layer are well known. To increase the efficiency of recombination of injected holes and electrons in the devices of the laminate type, the structure of the device and the process for forming the device have been studied.

Conventionally, aromatic diamine derivatives described in Patent Document 1 and aromatic fused ring diamine derivatives with fused rings described in Patent Document 2 have been known as hole transporting materials for the organic EL devices. Improving those aromatic amine derivatives, Patent Document 3 discloses the following Compound (A), and Patent Document 4 discloses an aromatic triamine compound represented by the following general formula (B). Further, Patent Document 5 discloses a naphthylene amine compound represented by the following general formula (C). Furthermore, tetramers of the aromatic amine such as disclosed in Patent Document 6 or Patent Document 7 are known as a highly heat resistant material.

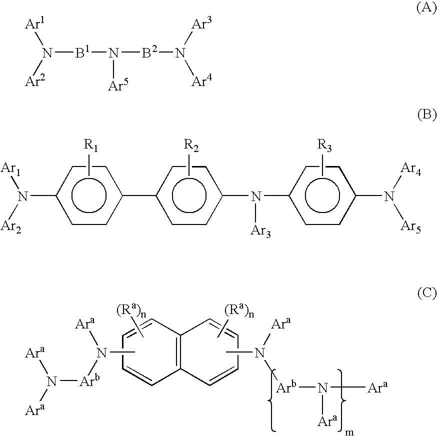

In the general formula (A), B¹ and B² each independently represents a substituted or unsubstituted biphenylene group.

Moreover, Patent Document 8 discloses a following general formula (D), Patent Document 9 discloses a tetraamine compound of a following Compound (E), Patent Document 10 discloses a following Compound (F) and Patent Document 11 discloses tetraamine compounds containing naphthalene of following Compounds (G) and (H).

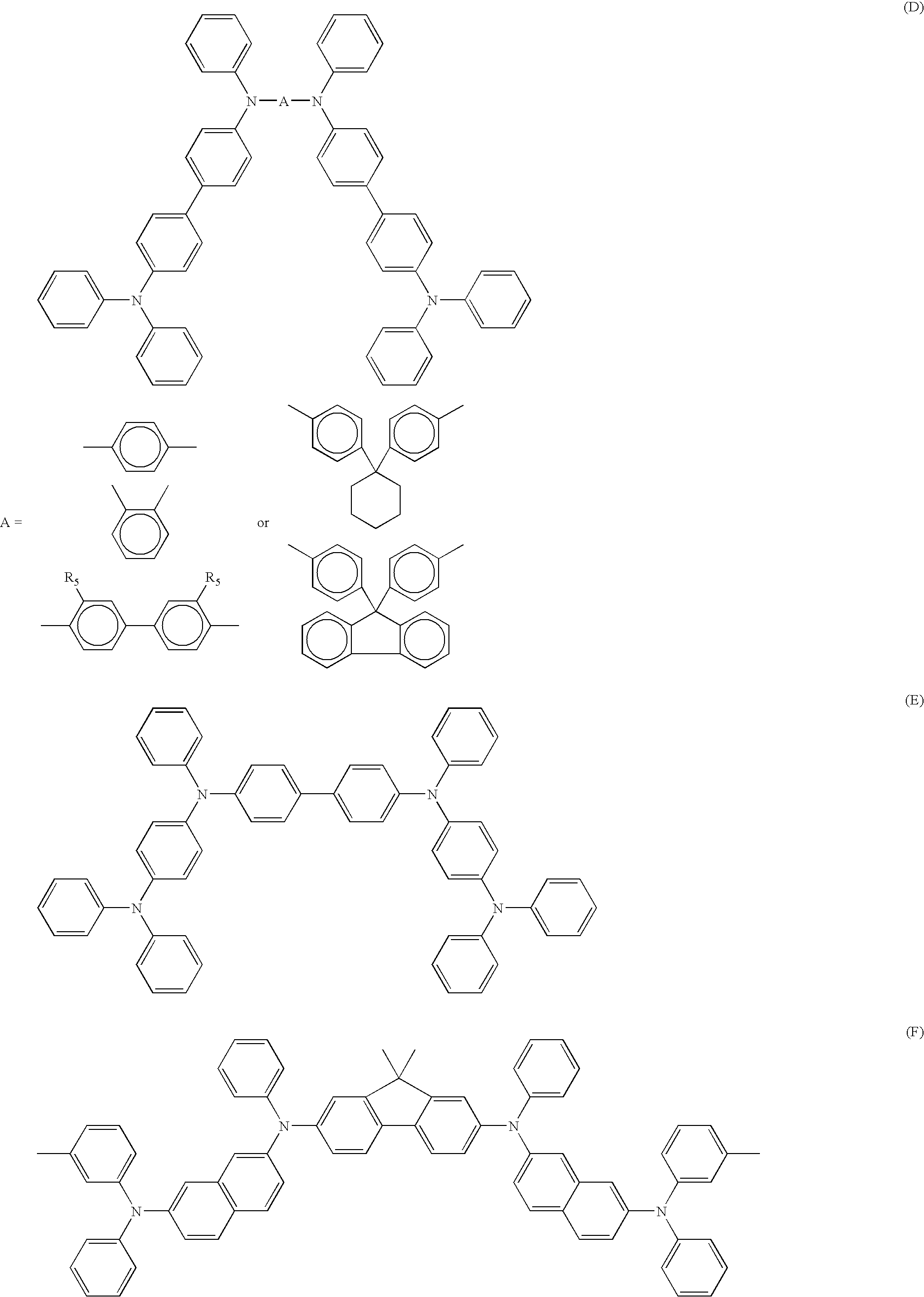

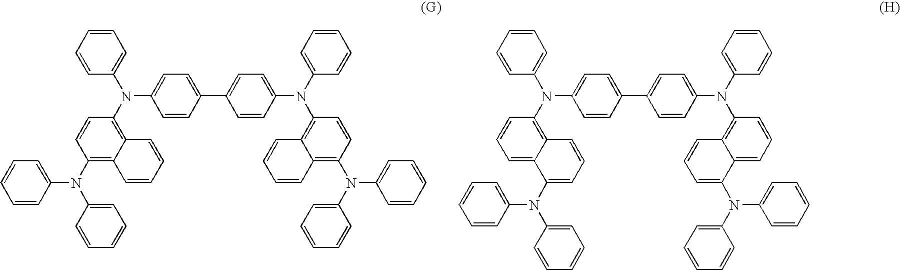

However, all the devices employing the above compounds for the hole transporting layer are not sufficient in their lifetime, driving voltage and efficiency of light emission. Therefore, low voltage driving, improving an efficiency of light emission and prolonged lifetime are eagerly desired.

Patent Document 1: U.S. Pat. No. 4,720,432

Patent Document 2: U.S. Pat. No. 5,061,569

Patent Document 3: Japanese Patent No. 3565870

Patent Document 4: Japanese Patent No. 3220867

Patent Document 5: JP 2003-133076A

Patent Document 6: Japanese Patent No. 3194657

Patent Document 7: Japanese Patent No. 3180802

Patent Document 8: Japanese Patent No. 3220950

Patent Document 9: JP 2000-156290A

Patent Document 10: JP 2001-226331A

Patent Document 11: JP 1999-236360A

### DISCLOSURE OF THE INVENTION

### Problems to be Solved by the Invention

The present invention has been made to overcome the above problems and has an object of providing a material containing an aromatic amine derivative for an organic EL device satisfying a reduction of its driving voltage and an enhance of its efficiency of light emission simultaneously, together with maintaining its long lifetime.

### Means for Solving the Problem

As a result of intensive researches and studies to overcome the above problems, the inventors have found that an employment of a material for the organic EL device containing an aromatic amine derivative represented by any one of the following general formulae (1), (2) and (5) enables to produce an organic EL device driven under a low electric voltage and with an enhanced efficiency of light emission while maintaining its long lifetime, thereby completing the present invention.

Namely, the present invention provides an aromatic amine derivative represented by any one of the following general formulae (1), (2) and (5).

An aromatic amine derivative represented by the following general formula (1): 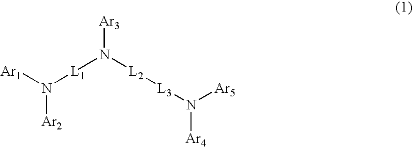
[In the formula, Ar₁ to Ar₅ each independently represents a substituted or unsubstituted aryl group having 6 to 50 ring carbon atoms.

L₁ to L₃ each independently represents a substituted or unsubstituted arylene group having 6 to 50 ring carbon atoms; with the proviso that at least one of L₁ to L₃ represents a substituted or unsubstituted arylene group which contains a fused aromatic ring having 6 to 50 ring carbon atoms.]

An aromatic amine derivative represented by the following general formula (2): 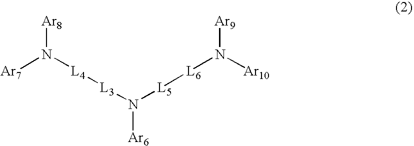
[In the formula, Ar₆ to Ar₁₀ represents a substituted or unsubstituted aryl group having 6 to 50 ring carbon atoms.

L₃ to L₆ represents a substituted or unsubstituted arylene group having 6 to 50 ring carbon atoms; with the proviso that at least one of L₃ to L₆ represents a substituted or unsubstituted fused aromatic ring group having 6 to 50 ring carbon atoms.]

An aromatic amine derivative represented by the following general formula (5): 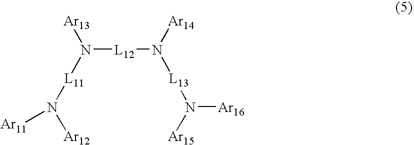
[In the formula, Ar₁₁ to Ar₁₆ each independently represents a substituted or unsubstituted aryl group having 6 to 50 ring carbon atoms.

L₁₁ and L₁₃ each independently represents a single bond or a substituted or unsubstituted arylene group having 6 to 50 ring carbon atoms.

L₁₂ is represented by the following general formula (6): 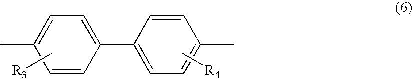
{In the formula, R₃ and R₄ each independently represents a substituent.}

At least one of L₁₁ and L₁₃ corresponds to a naphthylene derivative represented by the following general formula (7): 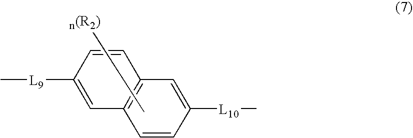
{R₂ represents a substituent, n represents an integer of 0 to 6; and

L₉ and L₁₀ each independently represents a single bond or a substituted or unsubstituted arylene group having 6 to 50 ring carbon atoms.}]

An organic EL device of the present invention is composed of one or more organic thin film layers including at least one light emitting layer and sandwiched between an anode and a cathode, wherein at least one of the organic thin film layers contains the aromatic amine derivative represented by any one of the above general formulae (1), (2) and (5) singly or as its mixture component.

### EFFECT OF THE INVENTION

An employment of a material for the organic EL device represented by any one of the following general formulae (1), (2) and (5) to any one of the organic thin film layers, preferably to a hole transporting region and/or a hole injecting region, more preferably to a hole transporting region and/or a hole injecting region enables to produce an organic EL device capable of emitting blue light, driven under a low electric voltage and exhibiting enhanced efficiency of light emission while maintaining its long lifetime.

### PREFERRED EMBODIMENT TO CARRY OUT THE INVENTION

The present invention provides an aromatic amine derivative represented by any one of the following general formulae (1), (2) and (5).

An aromatic amine derivative represented by the following general formula (1):

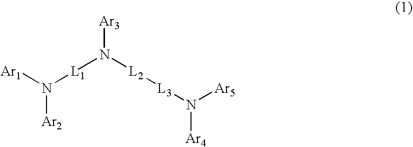

In the general formula (1), Ar₁ to Ar₅ each independently represents a substituted or unsubstituted aryl group having 6 to 50 ring carbon atoms. L₁ to L₃ each independently represents a substituted or unsubstituted arylene group having 6 to 50 ring carbon atoms; with the proviso that at least one of L₁ to L₃ represents a substituted or unsubstituted arylene group which contains a fused aromatic ring having 6 to 50 ring carbon atoms.

An aromatic amine derivative represented by the following general formula (2):

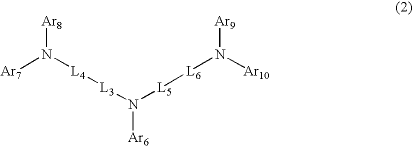

In the general formula (2), Ar₆ to Ar₁₀ represents a substituted or unsubstituted aryl group having 6 to 50 ring carbon atoms. L₃ to L₆ represents a substituted or unsubstituted arylene group having 6 to 50 ring carbon atoms; with the proviso that at least one of L₃ to L₆ represents a substituted or unsubstituted fused aromatic ring group having 6 to 50 ring carbon atoms.

Examples of the substituted or unsubstituted aryl group preferably include phenyl group, 1-naphthyl group, 2-naphthyl group, 1-anthracenyl group, 2-anthracenyl group, 9-anthracenyl group, 1-phenanthryl group, 2-phenanthryl group, 3-phenanthryl group, 4-phenanthryl group, 9-phenanthryl group, 1-naphthacenyl group, 2-naphthacenyl group, 9-naphthacenyl group, 1-pyrenyl group, 2-pyrenyl group, 4-pyrenyl group, 2-biphenylyl group, 3-biphenylyl group, 4-biphenylyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-tolyl group, m-tolyl group, p-tolyl group, p-t-butylphenyl group, p-(2-phenylpropyl)phenyl group, 3-methyl-2-naphthyl group, 4-methyl-1-naphthyl group, 4-methyl-1-anthryl group, 4′-methylbiphenylyl group, 4″-t-butyl-p-terphenyl-4-yl group, fluorenyl group, etc.

Preferable examples of the arylene group having 6 to 60 ring carbon atoms include phenylene group, biphenylene group, terphenylene group, quarterphenylene group, naphthylene group, anthracenylene group, phenanthrylene group, chrycenylene group, pyrenylene group, perilenylene group, fluorenylene group, etc. More preferable examples are phenylene group, biphenylene group, terphenylene group, fluorenylene group, naphthylene group, chrycenylene group, etc.

Examples of a substituted or unsubstituted fused aromatic group include naphthylene group, anthracenylene group, phenanthrylene group, crycenylene group, pyrenylene group, perilenylene, etc.; which are fused aromatic ring groups among the above arylene groups. Preferable examples are naphthylene group, phenanthrylene group, chrycenylene group and pyrenylene group.

In the foregoing general formula (2), at least one among L₃ to L₆ is preferably an aromatic amine derivative containing a substituted or unsubstituted naphthalene, and more preferably, an aromatic amine derivative represented by the following general formulae (3) and (4).

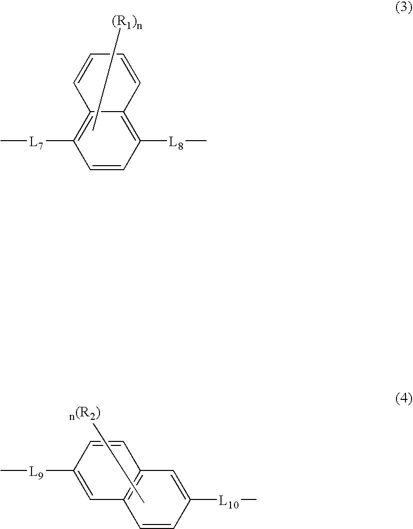

In the formulae (3) and (4), R₁ and R₂ each independently represents a substituent, and n represents an integer of 0 to 6. L₇ to L₁₀ represents a single bond or a substituted or unsubstituted arylene group having 6 to 50 ring carbon atoms. Specific examples of those are the same as described above.

Preferable substituents of Ar₁ to Ar₅ and L₃ to L₆ include alkyl group (alkyl group preferably having 1 to 20 carbon atoms, more preferably having 1 to 12 carbon atoms and particularly preferably having 1 to 8 carbon atoms; examples include methyl group, ethyl group, iso-propyl group, tert-butyl group, n-octyl group, n-decyl group, n-hexadecyl group, cyclopropyl group, cyclopentyl group, cyclohexyl group, etc.); alkenyl group (alkenyl group preferably having 2 to 20 carbon atoms, more preferably having 2 to 12 carbon atoms and particularly preferably having 2 to 8 carbon atoms; examples include vinyl group, allyl group, 2-butenyl group, 3-pentenyl group, etc.); alkynyl group (alkynyl group preferably having 2 to 20 carbon atoms, more preferably having 2 to 12 carbon atoms and particularly preferably having 2 to 8 carbon atoms; examples include propargyl group, 3-pentynyl group, etc.); amino group (amino group preferably having 0 to 20 carbon atoms, more preferably having 0 to 12 carbon atoms and particularly preferably having 0 to 6 carbon atoms; examples include amino group, methylamino group, dimethylamino group, diethylamino group, diphenylamino group, dibenzylamino group, etc.); alkoxy group (alkoxy group preferably having 1 to 20 carbon atoms, more preferably having 1 to 12 carbon atoms and particularly preferably having 1 to 8 carbon atoms; examples include methoxy group, ethoxy group, butoxy group, etc.); aryloxy group (aryloxy group preferably having 6 to 20 carbon atoms, more preferably having 6 to 16 carbon atoms and particularly preferably having 6 to 12 carbon atoms; examples include phenyloxy group, 2-naphthyloxy group, etc.); acyl group (acyl group preferably having 1 to 20 carbon atoms, more preferably having 1 to 16 carbon atoms and particularly preferably having 1 to 12 carbon atoms; examples include acetyl group, benzoyl group, formyl group, pivaloyl group, etc.); alkoxycarbonyl group (alkoxycarbonyl group preferably having 2 to 20 carbon atoms, more preferably having 2 to 16 carbon atoms and particularly preferably having 2 to 12 carbon atoms; examples include methoxycarbonyl group, ethoxycarbonyl group, etc.); aryloxycarbonyl group (aryloxycarbonyl group preferably having 7 to 20 carbon atoms, more preferably having 7 to 16 carbon atoms and particularly preferably having 7 to 10 carbon atoms; examples include phenyloxycarbonyl group, etc.); acyloxy group (acyloxy group preferably having 2 to 20 carbon atoms, more preferably having 2 to 16 carbon atoms and particularly preferably having 2 to 10 carbon atoms; examples include acetoxy group, benzoyloxy group, etc.); acylamino group (acylamino group preferably having 2 to 20 carbon atoms, more preferably having 2 to 16 carbon atoms and particularly preferably having 2 to 10 carbon atoms; examples include acetylamino group, benzoylamino group, etc.); alkoxycarbonylamino group (alkoxycarbonylamino group preferably having 2 to 20 carbon atoms, more preferably having 2 to 16 carbon atoms and particularly preferably having 2 to 12 carbon atoms; examples include methoxycarbonylamino group, etc.); aryloxycarbonylamino group (aryloxycarbonylamino group preferably having 7 to 20 carbon atoms, more preferably having 7 to 16 carbon atoms and particularly preferably having 7 to 12 carbon atoms; examples include phenyloxycarbonylamino group, etc.); sulfonylamino group (sulfonylamino group preferably having 1 to 20 carbon atoms, more preferably having 1 to 16 carbon atoms and particularly preferably having 1 to 12 carbon atoms; examples include methanesulfonylamino group, benzensulfonylamino group, etc.); sulfamoyl group (sulfamoyl group preferably having 0 to 20 carbon atoms, more preferably having 0 to 16 carbon atoms and particularly preferably having 0 to 12 carbon atoms; examples include sulfamoyl group, methylsulfamoyl group, dimethylsulfamoyl group, phenylsulfamoyl group, etc.); carbamoyl group (carbamoyl group preferably having 1 to 20 carbon atoms, more preferably having 1 to 16 carbon atoms and particularly preferably having 1 to 12 carbon atoms; examples include carbamoyl group, methylcarbamoyl group, diethylcarbamoyl group, phenylcarbamoyl group, etc.); alkylthio group (alkylthio group preferably having 1 to 20 carbon atoms, more preferably having 1 to 16 carbon atoms and particularly preferably having 1 to 12 carbon atoms; examples include methylthio group, ethylthio group, etc.); arylthio group (arylthio group preferably having 6 to 20 carbon atoms, more preferably having 6 to 16 carbon atoms and particularly preferably having 6 to 12 carbon atoms; examples include phenylthio group, etc.); sulfonyl group (sulfonyl group preferably having 1 to 20 carbon atoms, more preferably having 1 to 16 carbon atoms and particularly preferably having 1 to 12 carbon atoms; examples include mesyl group, tosyl group, etc.); sulfinyl group (sulfinyl group preferably having 1 to 20 carbon atoms, more preferably having 1 to 16 carbon atoms and particularly preferably having 1 to 12 carbon atoms; examples include methanesulfinyl group, benzenesulfinyl group, etc.); ureide group (ureide group preferably having 1 to 20 carbon atoms, more preferably having 1 to 16 carbon atoms and particularly preferably having 1 to 12 carbon atoms; examples include ureide group, methylureide group, phenylureide group, etc.); phosphoricamide group (phosphoricamide group preferably having 1 to 20 carbon atoms, more preferably having 1 to 16 carbon atoms and particularly preferably having 1 to 12 carbon atoms; examples include diethylphosphoricamide group, phenylphosphateamide group, etc.); hydroxy group; mercapto group; halogen atom (for example, fluorine atom, chlorine atom, bromine atom, iodine atom); cyano group; sulfo group; carboxyl group; nitro group; hydroxamicacid group; sulfino group; hydrazino group; imino group; heterocyclic group (heterocyclic group preferably having 1 to 30 carbon atoms, more preferably having 1 to 12 carbon atoms; examples of the hetero atom include nitrogen atom, oxygen atom, sulfur atom; specific examples of the heterocyclic group include imidazolyl, pyridyl, quinolyl, furyl, thienyl, piperidyl, morpholino, benzoxazolyl, benzimidazolyl, benzothiazolyl, carbazolyl, etc.); silyl group (silyl group preferably having 3 to 40 carbon atoms, more preferably having 3 to 30 carbon atoms and particularly preferably having 3 to 24 carbon atoms; examples include trimethylsilyl group, triphenylsilyl group, etc.); etc. Those substituents may be further substituted. Furthermore, when there are two or more substituents, the substituents may be the same with or different from each other. Moreover, in a case where it is possible, they may be bonded with each other to form a ring.

The triamine-based aromatic amine derivative represented by the foregoing general formula (1) or (2) may have a diarylamino group as a substituent, and preferably is an aromatic amine derivative represented by the following general formula (5):

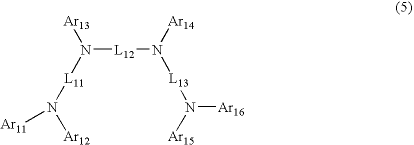

In the general formula (5), Ar₁₁ to Ar₁₆ each independently represents a substituted or unsubstituted aryl group having 6 to 50 ring carbon atoms. L₁₁ and L₁₃ each independently represents a single bond or a substituted or unsubstituted arylene group having 6 to 50 ring carbon atoms. L₁₂ is represented by the following general formula (6):

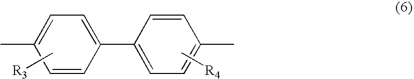

In the formula (6), R₃ and R₄ each independently represents a substituent.

At least one of L₁₁ and L₁₃ corresponds to a naphthylene derivative represented by the following general formula (7):

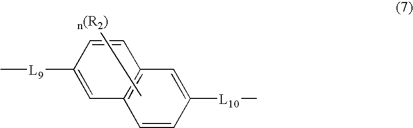

In the formula (7), R₂ represents a substituent, n represents an integer of 0 to 6; L₄ and L₁₀ each independently represents a single bond or a substituted or unsubstituted arylene group having 6 to 50 ring carbon atoms.

Specific examples of Ar₁₁ to Ar₁₆, and L₉ to L₁₃ are the same as described above.

Further, it is preferable that the derivative represented by the general formula (5) is the aromatic amine derivative having a structure of the following general formula (8):

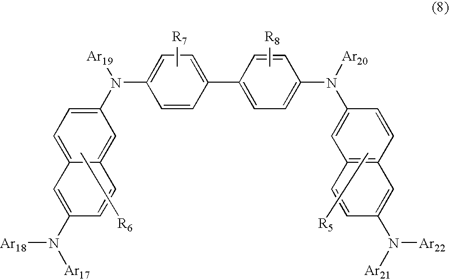

In the formula (8), Ar₁₇ to Ar₂₂ each independently represents a substituted or unsubstituted aryl group having 6 to 50 ring carbon atoms. R₅ to R₈ represent substituents.

Specific examples of Ar₁₇ to Ar₂₂, and R₅ to R₈ are the same as described above.

Examples of the compound represented by the general formulae (1) (2) and (5) will be shown below, however, the compounds of the present invention are not limited to them.

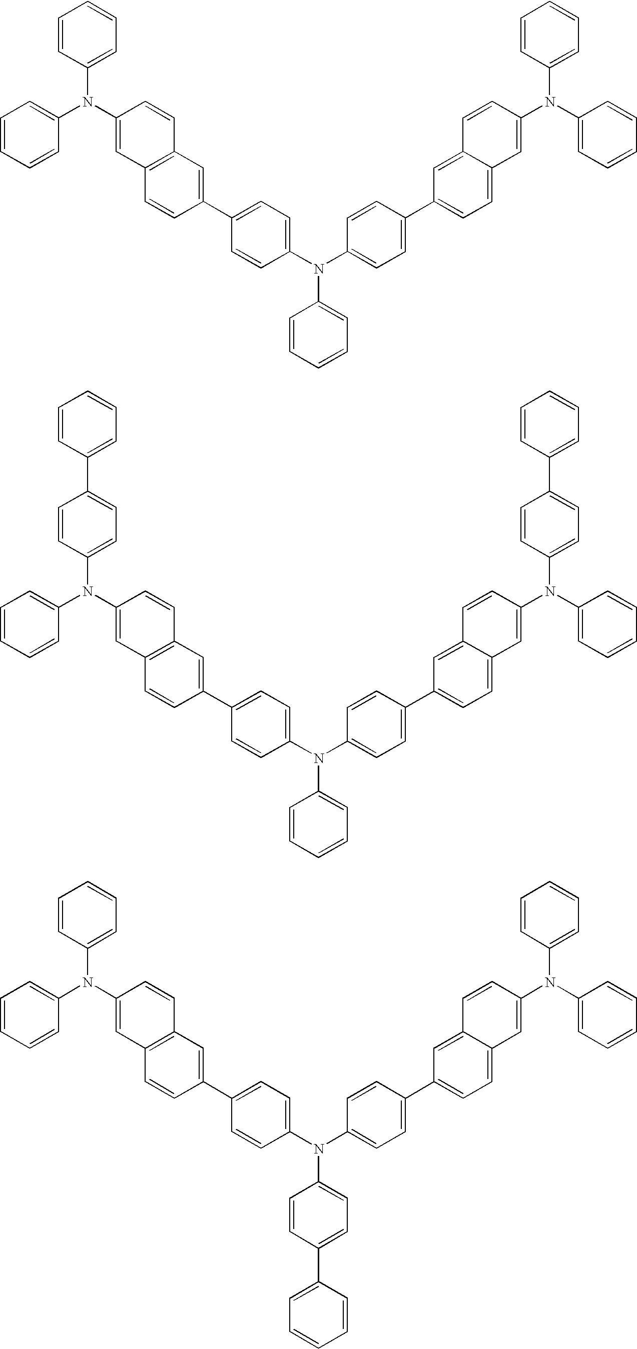

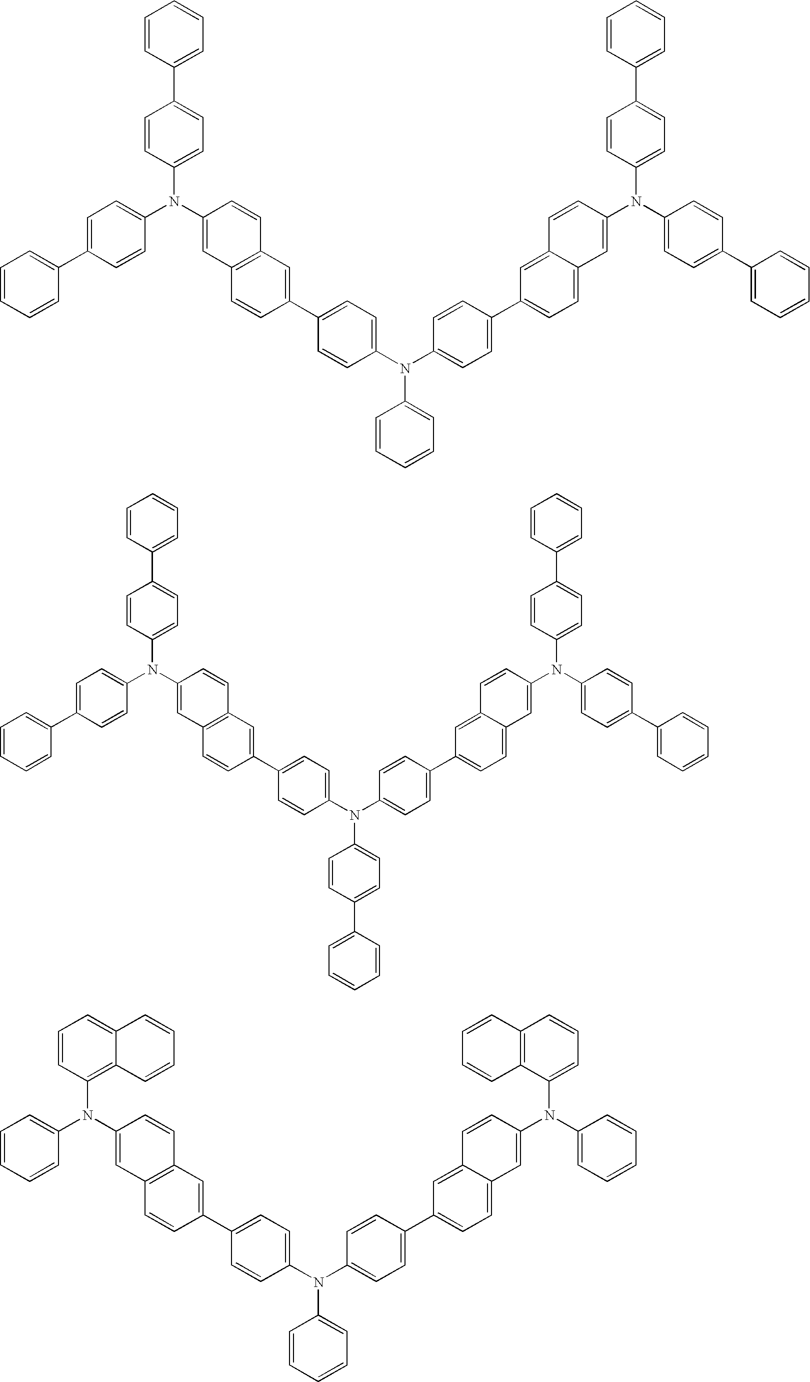

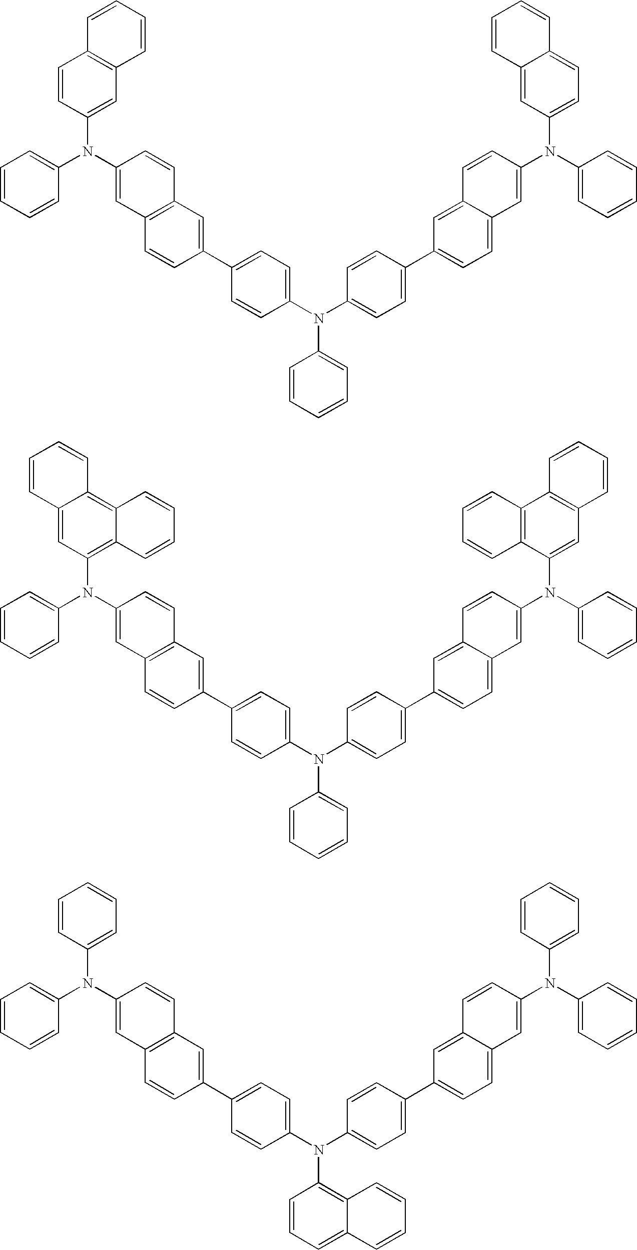

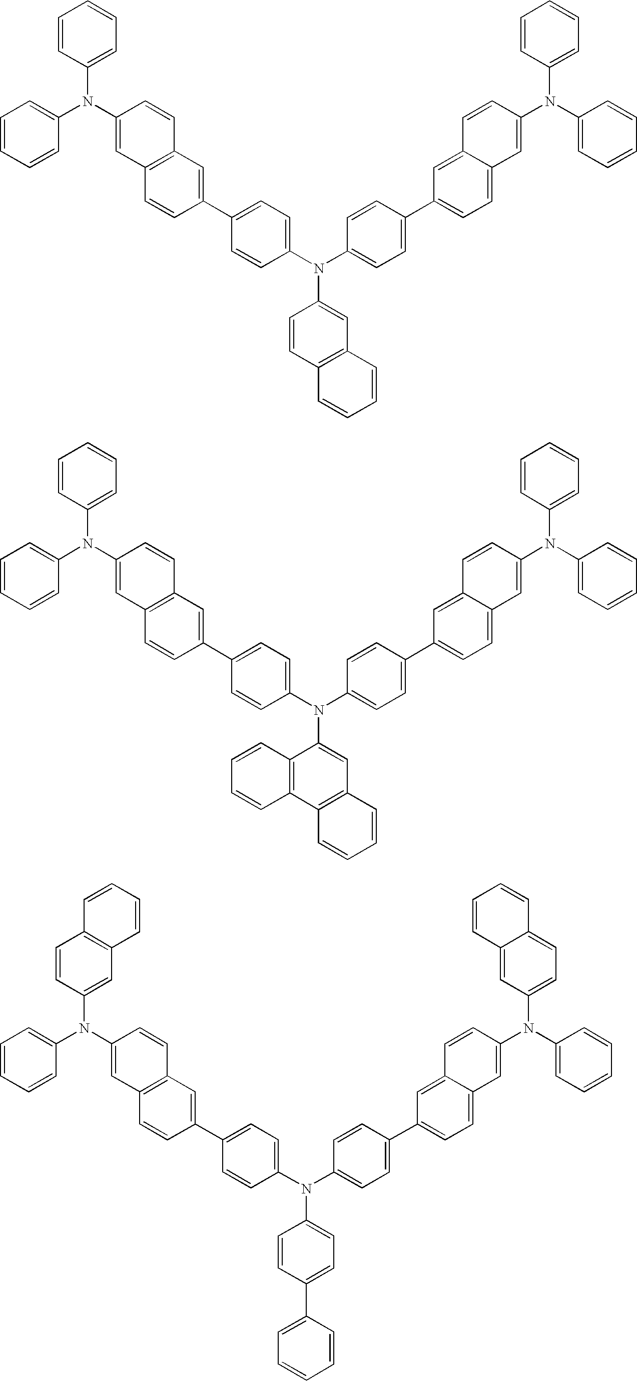

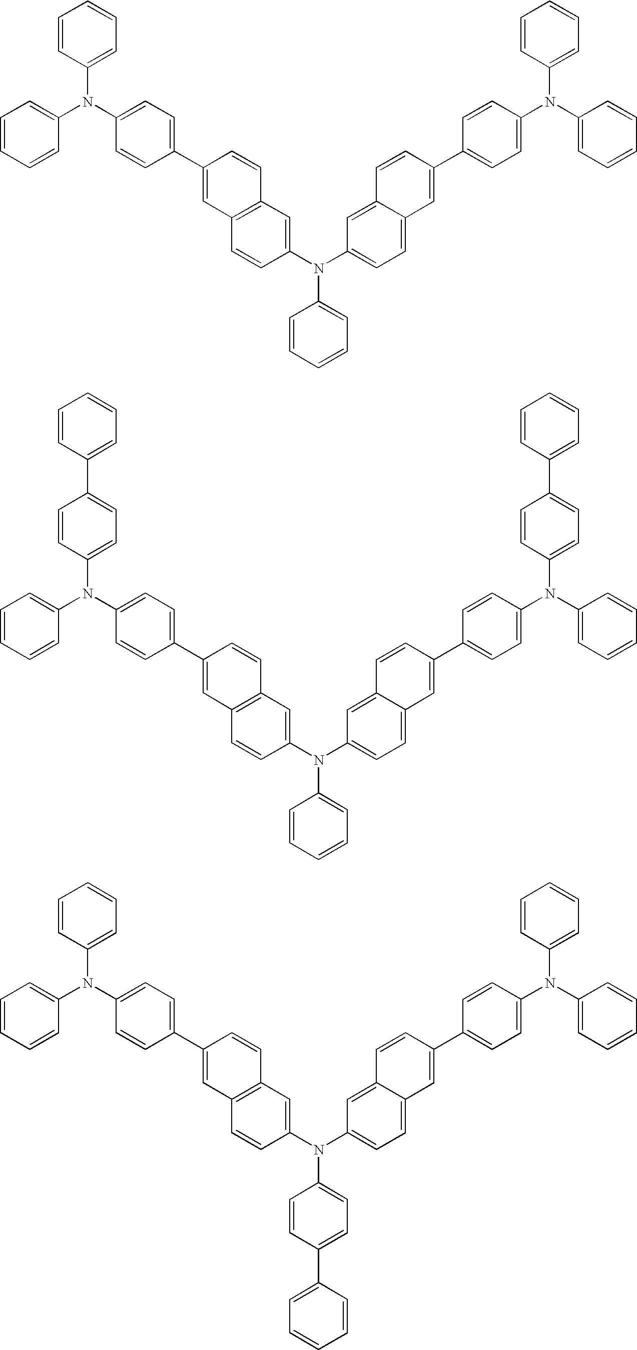

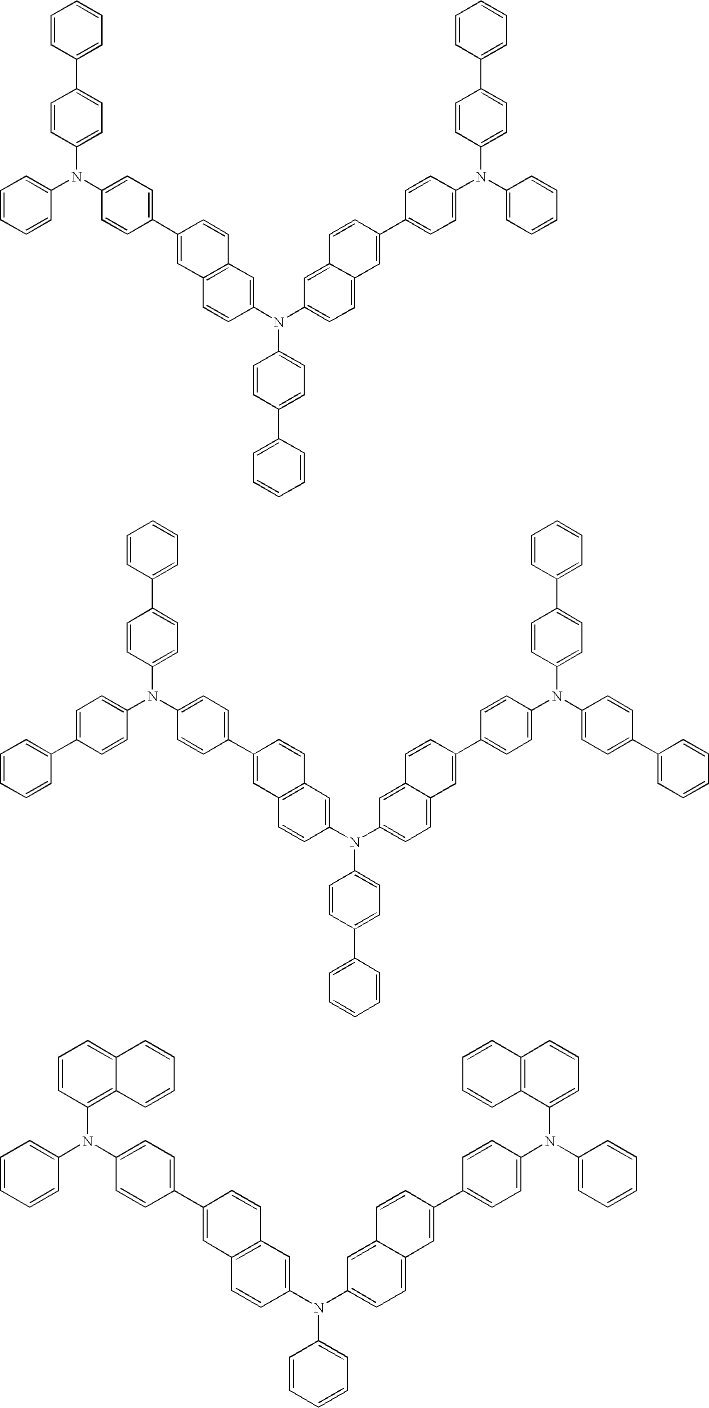

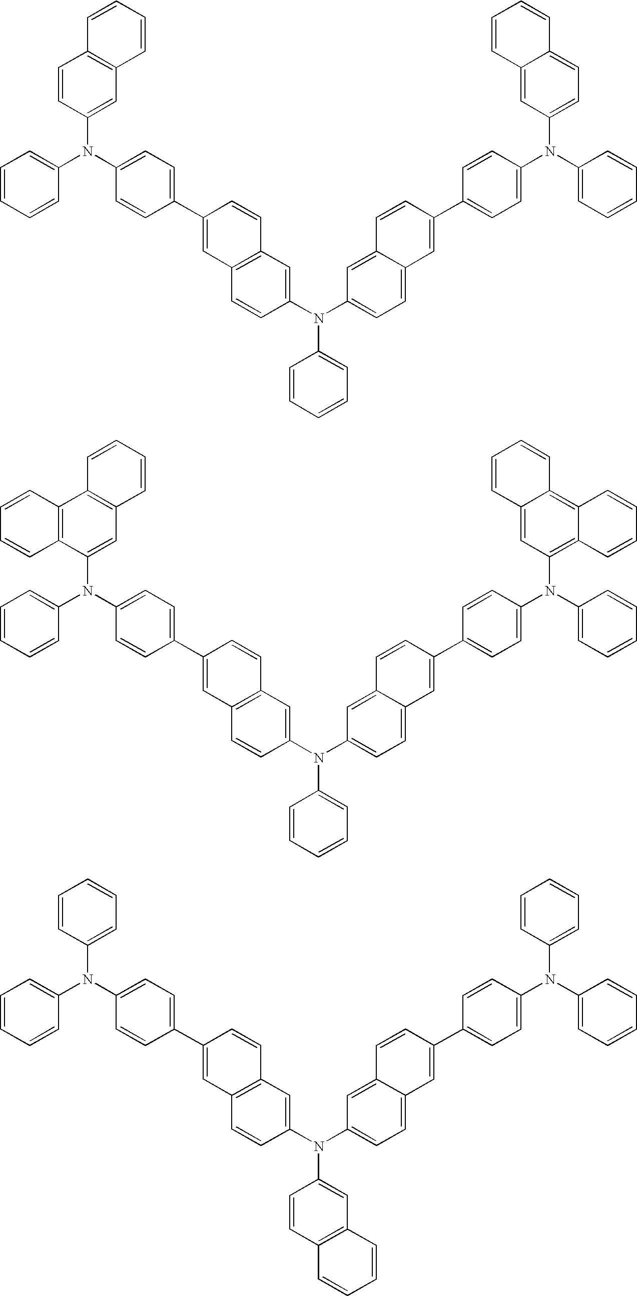

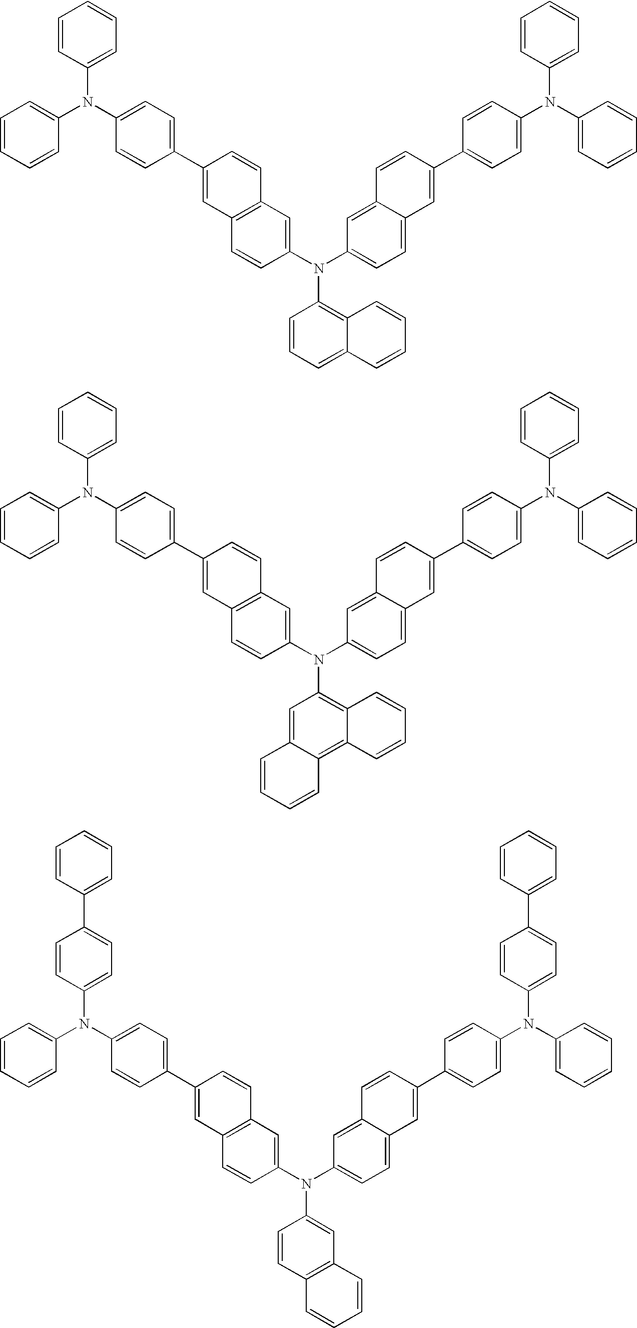

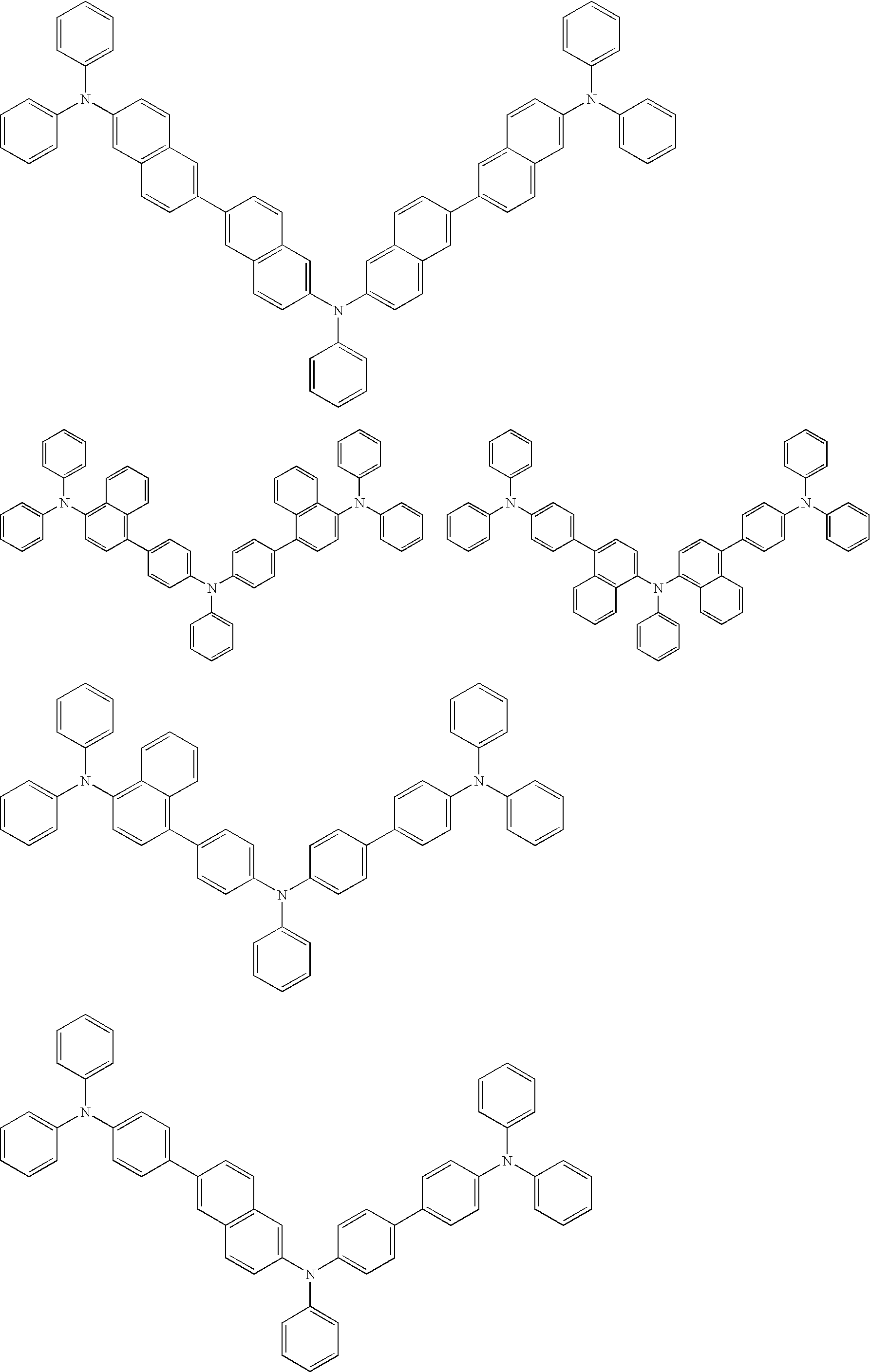

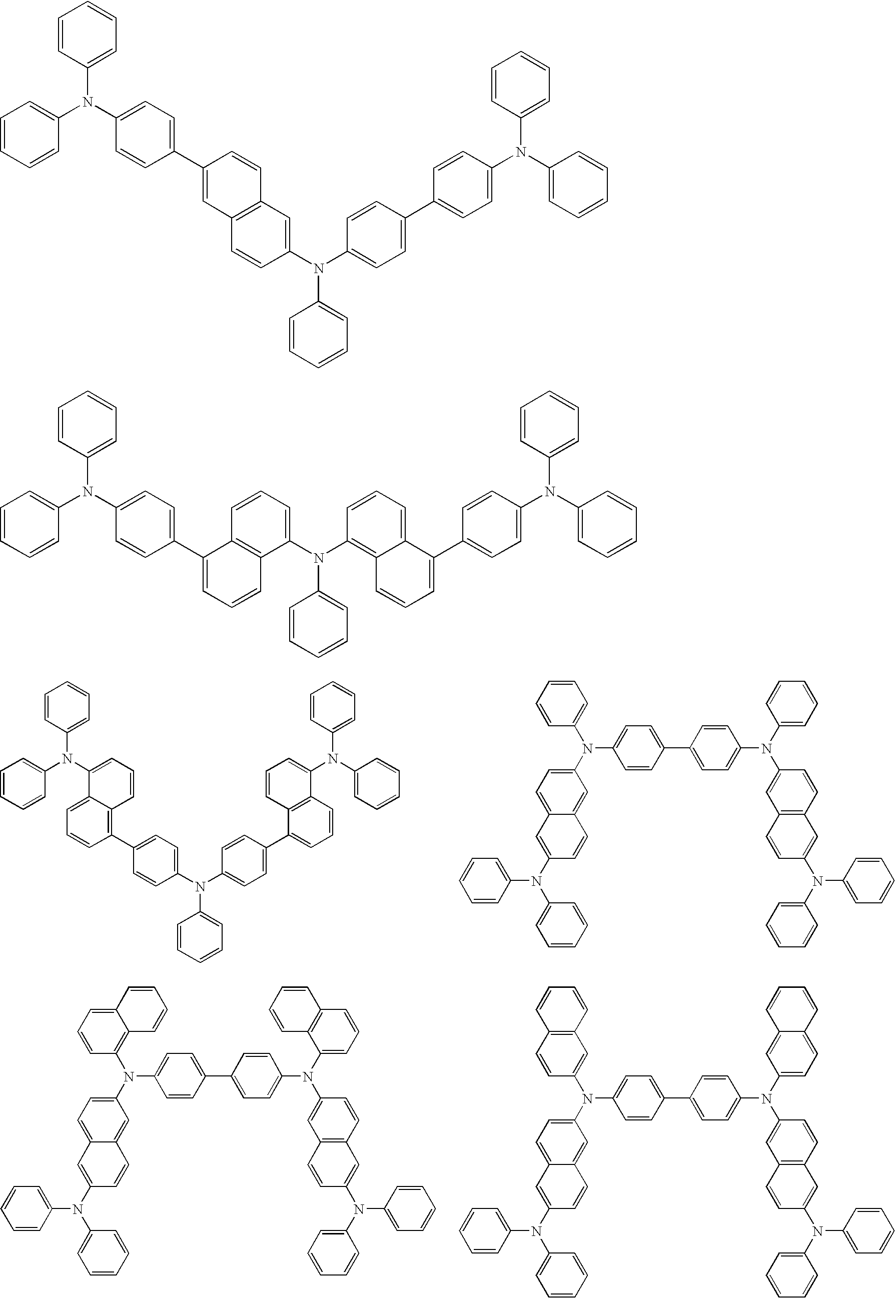

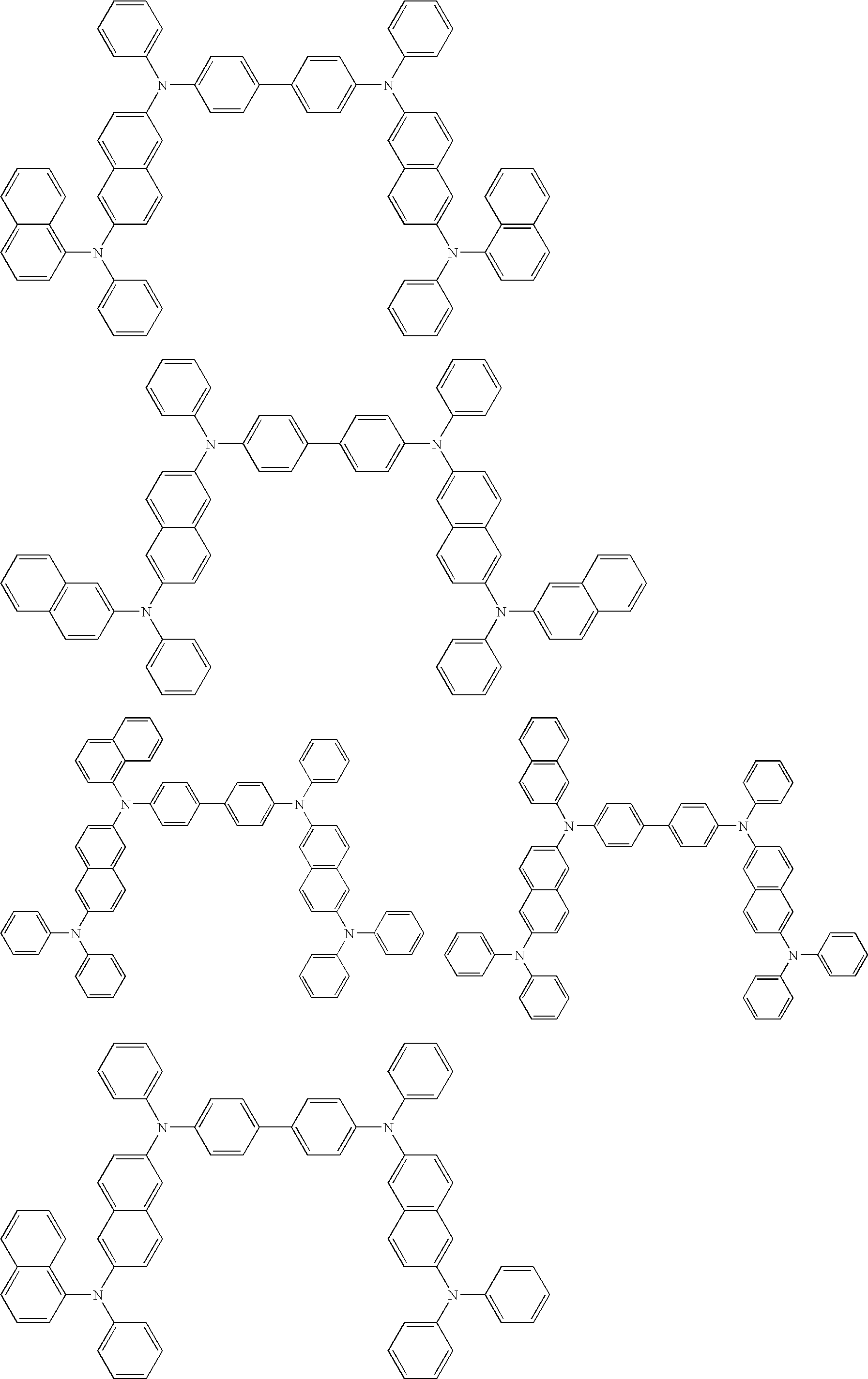

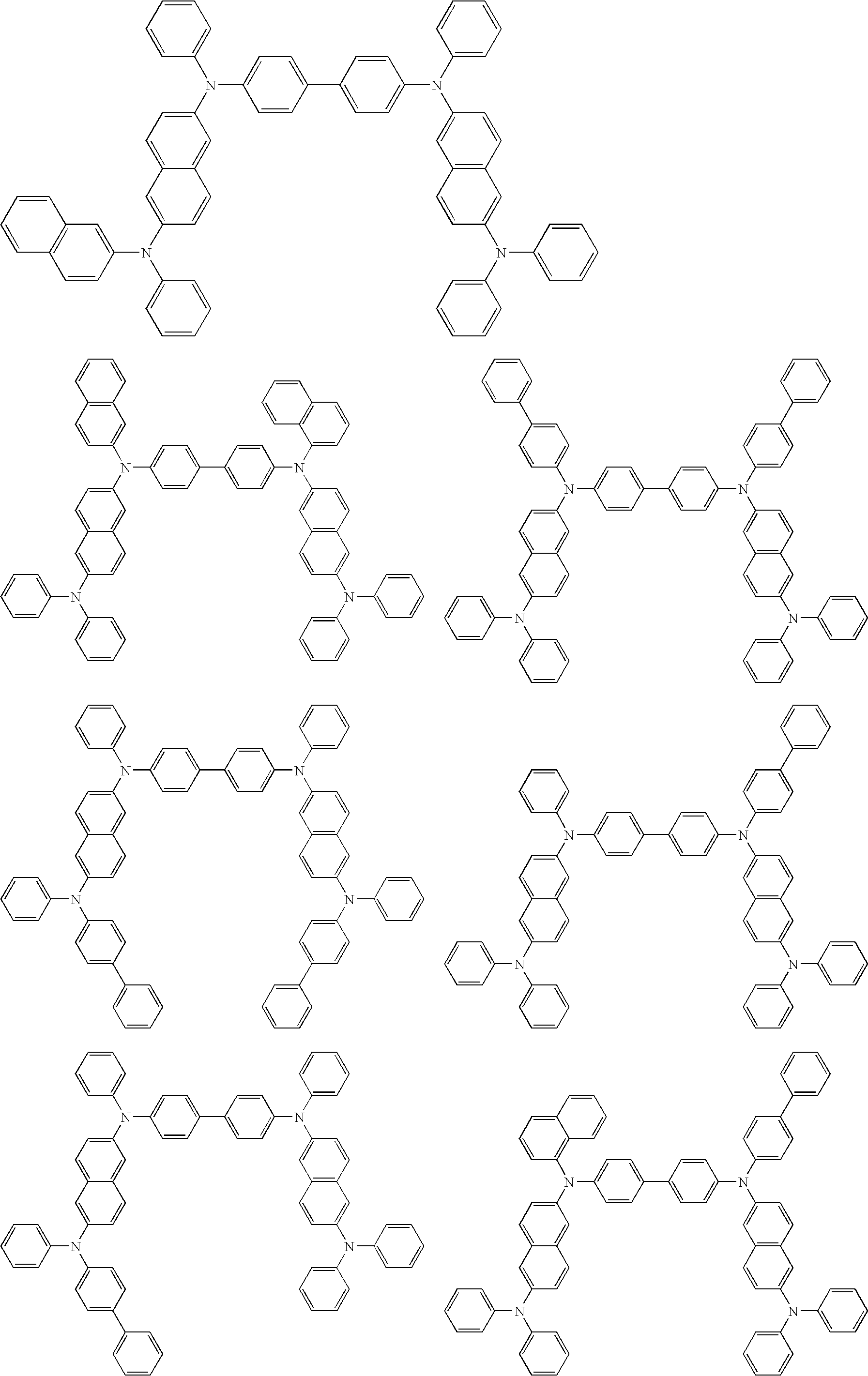

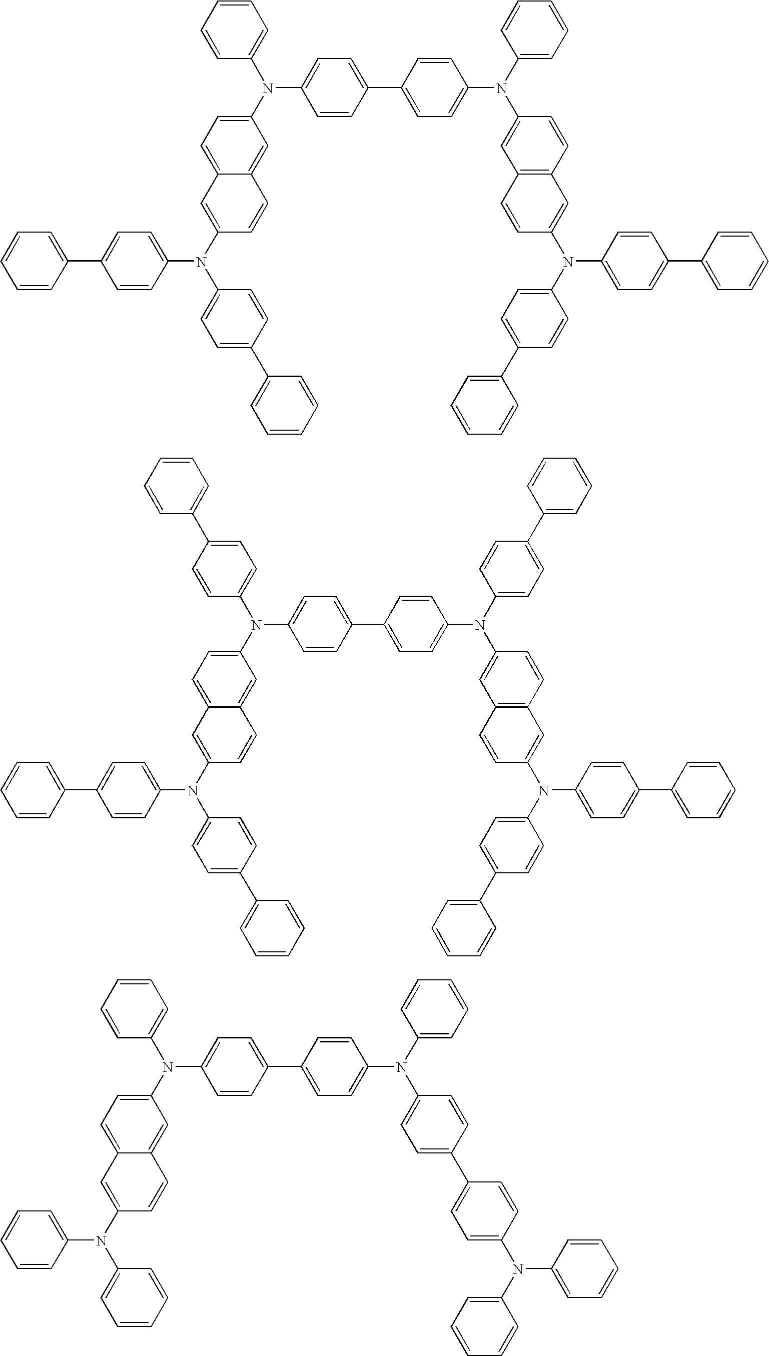

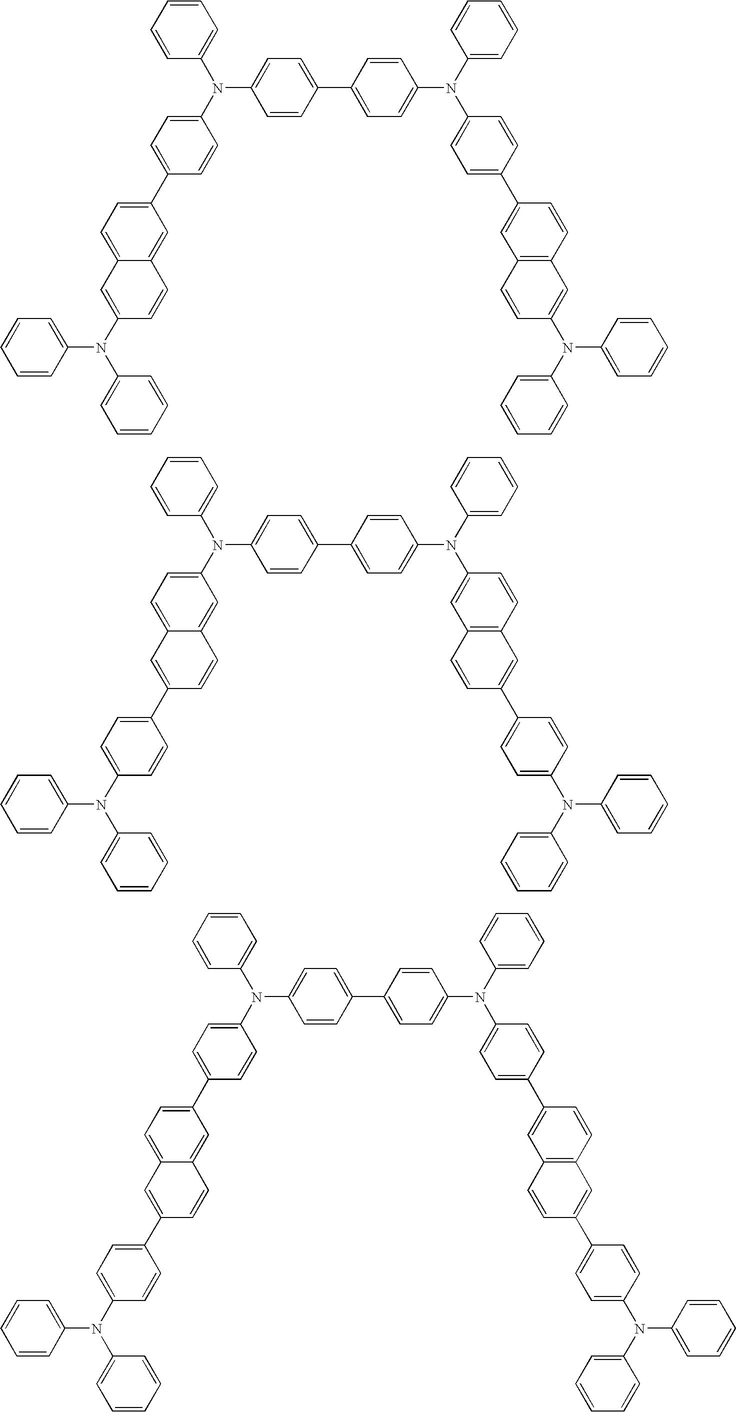

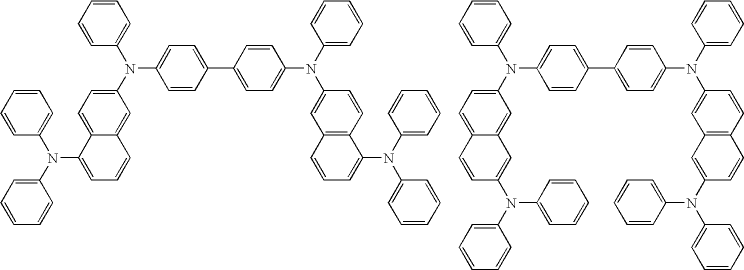

It is preferable that the aromatic amine derivative of the present invention is employed as a material for the organic electroluminescence devices. It is preferable that the aromatic amine derivative of the present invention is employed as a hole injecting material or a hole transporting material for the organic electroluminescence devices.

An organic EL device of the present invention is composed of one or more organic thin film layers including at least one light emitting layer sandwiched between an anode and a cathode, wherein at least one of the organic thin film layers contains the aromatic amine derivative represented by any one of the above general formulae (1), (2) and (5) singly or as its mixture component.

It is preferable for the organic EL device of the present invention, that the above organic thin film layers have a hole transporting region and/or a hole injecting region, and that any one of the above aromatic amine derivatives is used in the hole injecting region and/or the hole transporting region.

It is preferable for the organic EL device of the present invention, that the above organic thin film layers have a hole transporting layer and/or a hole injecting layer, and that any one of the above aromatic amine derivatives is used in the hole injecting layer and/or the hole transporting layer.

It is preferable that the organic EL device of the present invention emits blue light.

(I) Construction of the Organic EL Device

Following is a description regarding a typical device structure about the organic EL device of the present invention. Of course the present invention is not limited to them.

(1) An anode/a light emitting layer/a cathode

(2) An anode/a hole transporting layer/a light emitting layer/a cathode

(3) An anode/a light emitting layer/an electron transporting layer/a cathode

(4) An anode/a hole transporting layer/a light emitting layer/an electron transporting layer/a cathode

(5) An anode/a hole transporting layer/a light emitting layer/an adhesion improving layer/a cathode

(6) An anode/a hole injecting layer/a hole transporting layer/a light emitting layer/an electron transporting layer/a cathode

(7) An anode/a hole transporting layer/a light emitting layer/an electron transporting layer/an electron injecting layer/a cathode

(8) An anode/a hole injecting layer/a hole transporting layer/a light emitting layer/an electron transporting layer/an electron injecting layer/a cathode

(9) An anode/an insulating layer/a hole transporting layer/a light emitting layer/an electron transporting layer/a cathode

(10) An anode/a hole transporting layer/a light emitting layer/an electron transporting layer/an insulating layer/a cathode

(11) An anode/an inorganic semiconductor layer/an insulating layer/a hole transporting layer/a light emitting layer/an insulating layer/a cathode

(12) An anode/an insulating layer/a hole transporting layer/a light emitting layer/an electron transporting layer/an insulating layer/a cathode

(13) An anode/a hole injecting layer/a hole transporting layer/a light emitting layer/an electron transporting layer/an insulating layer/a cathode

(14) An anode/an insulating layer/a hole injecting layer/a hole transporting layer/a light emitting layer/an electron transporting layer/an electron injecting layer/a cathode

(15) An anode/an insulating layer/a hole injecting layer/a hole transporting layer/a light emitting layer/an electron transporting layer/an electron injecting layer/an insulating layer/a cathode

Among the above constructions, constructions (4), (6), (7), (8), (12), (13) and (15) are usually preferable.

Although the material for the organic EL device of the present invention may be employed in any of the above organic thin layers in the organic EL devices, it is contained preferably in a hole transporting region and/or a hole injecting region, and particularly preferably in the hole transporting layer.

(II) Light-Transmitting Substrate

In general, the organic EL device is fabricated on a light-transmitting substrate. The light-transmitting substrate is a substrate for supporting the organic EL device and preferably a flat and smooth substrate having a light transmittance of 50% or greater to visible light of 400 to 700 nm.

As the light-transmitting substrate, for example, glass plate and synthetic resin plate are advantageously employed. Specific examples of the glass plate include soda lime glass, glass containing barium and strontium, lead glass, aluminosilicate glass, borosilicate glass, barium borosilicate glass and quartz. Specific examples of the synthetic resin plate include plate made of polycarbonate resins, acrylic resins, polyethylene telephthalate resins, polyether sulfide resins and polysulfone resins.

(III) Anode

The anode in the organic EL device of the present invention has a function of injecting holes into a hole transporting layer or a light emitting layer, and it is effective that the anode has a work function of 4.5 eV or greater. Specific examples of the material for the anode include indium tin oxide alloy (ITO), tin oxide (NESA), indium-zinc oxide alloy (IZO), gold, silver, platinum, copper, lanthanoid, etc.

Those materials may be employed alone, however, alloys of those materials each other or a material made by adding other element may be employed by selecting appropriately.

The anode can be prepared by forming a thin film of the electrode material described above in accordance with a process such as the vapor deposition process and the sputtering process.

When the light emitted from the light emitting layer is obtained through the anode, it is preferable that the anode has a transmittance of the emitted light greater than 10%. It is also preferable that the sheet resistivity of the anode is several hundreds Ω/□ or smaller. The thickness of the anode is, in general, selected usually in the range of from 10 nm to 1 μm and preferably in the range of from 10 to 200 nm.

(IV) Light Emitting Layer

In the organic EL device of the present invention, the light emitting layer combines the following functions. Namely,

(1) The injecting function: the function of injecting holes from the anode or the hole injecting layer and injecting electrons from the cathode or the electron injecting layer when an electric field is applied;

(2) The transporting function: the function of transporting the injected charges (electrons and holes) by the force of the electric field; and

(3) The light emitting function: the function of providing the field for recombination of electrons and holes and promote the recombination to emit light.

Although there may be a difference between the capability of the holes being injected and the capability of the electrons being injected, and although there may be difference between the transporting functions expressed by mobilities of the holes and the electrons, either one of the charges is preferable to be transferred.

As the process for forming the light emitting layer, a well-known process such as the vapor deposition process, the spin coating process and the LB process can be employed. It is particularly preferable for the light emitting layer to be a molecular deposit film.

The molecular deposit film is a thin film formed by the deposition of a material compound in the gas phase or a thin film formed by the solidification of a material compound in a solution or liquid phase. In general, the molecular deposit film can be distinguished from the thin film formed in accordance with the LB process (the molecular accumulation film) based on the differences in the aggregation structure and higher order structures and functional differences caused by these structural differences.

In addition, as disclosed in JP 57-51781A, the light emitting layer can also be formed by dissolving a binder such as a resin and the material compounds into a solvent to prepare a solution, followed by forming a thin film from the prepared solution in accordance with the spin coating process or the like.

As for the material used for the light emitting layer, well-known light emitting materials with a prolonged lifetime can be employed, and a material represented by the following general formula (1) may be employed as the light emitting material:

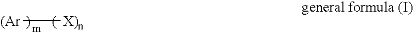

In the formula, Ar represents an aromatic ring having 6 to 50 ring carbon atoms or a heteroaromatic ring having 5 to 50 ring atoms.

Specific examples include phenyl ring, naphthyl ring, anthracene ring, biphenylene ring, azulene ring, acenaphthylene ring, fluorene ring, phenanthrene ring, fluoranthene ring, acephenanthrene ring, triphenylene ring, pyrene ring, chrysene ring, benzanthracene ring, naphthacene ring, picene ring, perylene ring, pentaphene ring, pentacene ring, tetraphenylene ring, hexaphene ring, hexacene ring, rubicene ring, coronene ring, trinaphtylene ring, pyrrole ring, indole ring, carbazole ring, imidazole ring, benzimidazole ring, oxadiazole ring, triazole ring, pyridine ring, quinoxaline ring, quinoline ring, pyrimidine ring, triazine ring, thiophene ring, benzothiophene ring, thianthrene ring, furan ring, benzofuran ring, pyrazole ring, pyrazine ring, pyridazine ring, indolizine ring, quinazoline ring, phenanthroline ring, silole ring, benzsilole ring, etc.

Preferable examples are phenyl ring, naphthyl ring, anthracene ring, acenaphthylene ring, fluorene ring, phenanthrene ring, fluoranthene ring, triphenylene ring, pyrene ring, chrysene ring, benzanthracene ring and perylene ring.

X represents a substituent.

Specifically, it corresponds to a substituted or unsubstituted aromatic group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted alkoxy group having 1 to 50 carbon atoms, a substituted or unsubstituted aralkyl group having 1 to 50 carbon atoms, a substituted or unsubstituted aryloxy group having 5 to 50 ring atoms, a substituted or unsubstituted arylthio group having 5 to 50 ring atoms, a substituted or unsubstituted carboxyl group having 1 to 50 carbon atoms, substituted or unsubstituted styryl group, a halogen atom, a cyano group, a nitro group or a hydroxyl group, etc.

Examples of the substituted or unsubstituted aromatic group having 6 to 50 ring carbon atoms represented by X include phenyl group, 1-naphthyl group, 2-naphthyl group, 1-anthryl group, 2-anthryl group, 9-anthryl group, 1-phenanthryl group, 2-phenanthryl group, 3-phenanthryl group, 4-phenanthryl group, 9-phenanthryl group, 1-naphthacenyl group, 2-naphthacenyl group, 9-naphthacenyl group, 1-pyrenyl group, 2-pyrenyl group, 4-pyrenyl group, 2-biphenylyl group, 3-biphenylyl group, 4-biphenylyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-tolyl group, m-tolyl group, p-tolyl group, p-t-butylphenyl group, p-(2-phenylpropyl)phenyl group, 3-methyl-2-naphthyl group, 4-methyl-1-naphthyl group, 4-methyl-1-anthryl group, 4′-methylbiphenylyl group, 4″-t-butyl-p-terphenyl-4-yl group, 2-fluorenyl group, 9,9-dimethyl-2-fluorenyl group, 3-fluoranthenyl group, etc.

Preferable examples include phenyl group, 1-naphthyl group, 2-naphthyl group, 9-phenanthryl group, 1-naphthacenyl group, 2-naphthacenyl group, 9-naphthacenyl group, 1-pyrenyl group, 2-pyrenyl group, 4-pyrenyl group, 2-biphenyl group, 3-biphenylyl group, 4-biphenylyl group, o-tolyl group, m-tolyl group, p-tolyl group, p-t-butylphenyl group, 2-fluorenyl group, 9,9-dimethyl-2-fluorenyl group, 3-fluoranthenyl group, etc.

Examples of the substituted or unsubstituted aromatic heterocyclic group having 5 to 50 ring atoms represented by X include 1-pyrrolyl group, 2-pyrrolyl group, 3-pyrrolyl group, pyradinyl group, 2-pyridinyl group, 3-pyridinyl group, 4-pyridinyl group, 1-indolyl group, 2-indolyl group, 3-indolyl group, 4-indolyl group, 5-indolyl group, 6-indolyl group, 7-indolyl group, 1-isoindolyl group, 2-isoindolyl group, 3-isoindolyl group, 4-isoindolyl group, 5-isoindolyl group, 6-isoindolyl group, 7-isoindolyl group, 2-furyl group, 3-furyl group, 2-benzofuranyl group, 3-benzofuranyl group, 4-benzofuranyl group, 5-benzofuranyl group, 6-benzofuranyl group, 7-benzofuranyl group, 1-isobenzofuranyl group, 3-isobenzofuranyl group, 4-isobenzofuranyl group, 5-isobenzofuranyl group, 6-isobenzofuranyl group, 7-isobenzofuranyl group, quinolyl group, 3-quinolyl group, 4-quinolyl group, 5-quinolyl group, 6-quinolyl group, 7-quinolyl group, 8-quinolyl group, 1-isoquinolyl group, 3-isoquinolyl group, 4-isoquinolyl group, 5-isoquinolyl group, 6-isoquinolyl group, 7-isoquinolyl group, 8-isoquinolyl group, 2-quinoxanyl group, 5-quinoxanyl group, 6-quinoxanyl group, 1-carbazolyl group, 2-carbazolyl group, 3-carbazolyl group, 4-carbazolyl group, 9-carbazolyl group, 1-phenanthridinyl group, 2-phenanthridinyl group, 3-phenanthridinyl group, 4-phenanthridinyl group, 6-phenanthridinyl group, 7-phenanthridinyl group, 8-phenanthridinyl group, 9-phenanthridinyl group, 10-phenanthridinyl group, 1-acridinyl group, 2-acridinyl group, 3-acridinyl group, 4-acridinyl group, 9-acridinyl group, 1,7-phenanthrolin-2-yl group, 1,7-phenanthrolin-3-yl group, 1,7-phenanthrolin-4-yl group, 1,7-phenanthrolin-5-yl group, 1,7-phenanthrolin-6-yl group, 1,7-phenanthrolin-8-yl group, 1,7-phenanthrolin-9-yl group, 1,7-phenanthrolin-10-yl group, 1,8-phenanthrolin-2-yl group, 1,8-phenanthrolin-3-yl group, 1,8-phenanthrolin-4-yl group, 1,8-phenanthrolin-5-yl group, 1,8-phenanthrolin-6-yl group, 1,8-phenanthrolin-7-yl group, 1,8-phenanthrolin-9-yl group, 1,8-phenanthrolin-10-yl group, 1,9-phenanthrolin-2-yl group, 1,9-phenanthrolin-3-yl group, 1,9-phenanthrolin-4-yl group, 1,9-phenanthrolin-5-yl group, 1,9-phenanthrolin-6-yl group, 1,9-phenanthrolin-7-yl group, 1,9-phenanthrolin-8-yl group, 1,9-phenanthrolin-10-yl group, 1,10-phenanthrolin-2-yl group, 1,10-phenanthrolin-3-yl group, 1,10-phenanthrolin-4-yl group, 1,10-phenanthrolin-5-yl group, 2,9-phenanthrolin-1-yl group, 2,9-phenanthrolin-3-yl group, 2,9-phenanthrolin-4-yl group, 2,9-phenanthrolin-5-yl group, 2,9-phenanthrolin-6-yl group, 2,9-phenanthrolin-7-yl group, 2,9-phenanthrolin-8-yl group, 2,9-phenanthrolin-10-yl group, 2,8-phenanthrolin-1-yl group, 2,8-phenanthrolin-3-yl group, 2,8-phenanthrolin-4-yl group, 2,8-phenanthrolin-5-yl group, 2,8-phenanthrolin-6-yl group, 2,8-phenanthrolin-7-yl group, 2,8-phenanthrolin-9-yl group, 2,8-phenanthrolin-10-yl group, 2,7-phenanthrolin-1-yl group, 2,7-phenanthrolin-3-yl group, 2,7-phenanthrolin-4-yl group, 2,7-phenanthrolin-5-yl group, 2,7-phenanthrolin-6-yl group, 2,7-phenanthrolin-8-yl group, 2,7-phenanthrolin-9-yl group, 2,7-phenanthrolin-10-yl group, 1-phenazinyl group, 2-phenazinyl group, 1-phenothiazinyl group, 2-phenothiazinyl group, 3-phenothiazinyl group, 4-phenothiazinyl group, 10-phenothiazinyl group, 1-phenoxazinyl group, 2-phenoxazinyl group, 3-phenoxazinyl group, 4-phenoxazinyl group, 10-phenoxazinyl group, 2-oxazolyl group, 4-oxazolyl group, 5-oxazolyl group, 2-oxadiazolyl group, 5-oxadiazolyl group, 3-furazanyl group, 2-thienyl group, 3-thienyl group, 2-methylpyrrol-1-yl group, 2-methylpyrrol-3-yl group, 2-methylpyrrol-4-yl group, 2-methylpyrrol-5-yl group, 3-methylpyrrol-1-yl group, 3-methylpyrrol-2-yl group, 3-methylpyrrol-4-yl group, 3-methylpyrrol-5-yl group, 2-t-butylpyrrol-4-yl group, 3-(2-phenylpropyl)pyrrol-1-yl group, 2-methyl-1-indolyl group, 4-methyl-1-indolyl group, 2-methyl-3-indolyl group, 4-methyl-3-indolyl group, 2-t-butyl-1-indolyl group, 4-t-butyl-1-indolyl group, 2-t-butyl-3-indolyl group, 4-t-butyl-3-indolyl group, etc.

Examples of the substituted or unsubstituted alkyl group having 1 to 50 carbon atoms represented by X include methyl group, ethyl group, propyl group, isopropyl group, n-butyl group, s-butyl group, isobutyl group, t-butyl group, n-pentyl group, n-hexyl group, n-heptyl group, n-octyl group, hydroxymethyl group, 1-hydroxyethyl group, 2-hydroxyethyl group, 2-hydroxyisobutyl group, 1,2-dihydroxyethyl group, 1,3-dihydroxyisopropyl group, 2,3-dihydroxy-t-butyl group, 1,2,3-trihydroxypropyl group, chloromethyl group, 1-chloroethyl group, 2-chloroethyl group, 2-chloroisobutyl group, 1,2-dichloroethyl group, 1,3-dichloroisopropyl group, 2,3-dichloro-t-butyl group, 1,2,3-trichloropropyl group, bromomethyl group, 1-bromoethyl group, 2-bromoethyl group, 2-bromoisobutyl group, 1,2-dibromoethyl group, 1,3-dibromoisopropyl group, 2,3-dibromo-t-butyl group, 1,2,3-tribromopropyl group, iodomethyl group, 1-iodoethyl group, 2-iodoethyl group, 2-iodoisobutyl group, 1,2-diiodoethyl group, 1,3-diiodoisopropyl group, 2,3-diiodo-t-butyl group, 1,2,3-triiodopropyl group, aminomethyl group, 1-aminoethyl group, 2-aminoethyl group, 2-aminoisobutyl group, 1,2-diaminoethyl group, 1,3-diaminoisopropyl group, 2,3-diamino-t-butyl group, 1,2,3-triaminopropyl group, cyanomethyl group, 1-cyanoethyl group, 2-cyanoethyl group, 2-cyanoisobutyl group, 1,2-dicyanoethyl group, 1,3-dicyanoisopropyl group, 2,3-dicyano-t-butyl group, 1,2,3-tricyanopropyl group, nitromethyl group, 1-nitroethyl group, 2-nitroethyl group, 2-nitroisobutyl group, 1,2-dinitroethyl group, 1,3-dinitroisopropyl group, 2,3-dinitro-t-butyl group, 1,2,3-trinitropropyl group, cyclopropyl group, cyclobutyl group, cyclopentyl group, cyclohexyl group, 4-methylcyclohexyl group, 1-adamanthyl group, 2-adamanthyl group, 1-norbornyl group, 2-norbornyl group, etc.

The substituted or unsubstituted alkoxyl group represented by X is a group represented by —OY. Examples of the group represented by Y include methyl group, ethyl group, propyl group, isopropyl group, n-butyl group, s-butyl group, isobutyl group, t-butyl group, n-pentyl group, n-hexyl group, n-heptyl group, n-octyl group, hydroxymethyl group, 1-hydroxyethyl group, 2-hydroxyethyl group, 2-hydroxyisobutyl group, 1,2-dihydroxyethyl group, 1,3-dihydroxyisopropyl group, 2,3-dihydroxy-t-butyl group, 1,2,3-trihydroxypropyl group, chloromethyl group, 1-chloroethyl group, 2-chloroethyl group, 2-chloroisobutyl group, 1,2-dichloroethyl group, 1,3-dichloroisopropyl group, 2,3-dichloro-t-butyl group, 1,2,3-trichloropropyl group, bromomethyl group, 1-bromoethyl group, 2-bromoethyl group, 2-bromoisobutyl group, 1,2-dibromoethyl group, 1,3-dibromoisopropyl group, 2,3-dibromo-t-butyl group, 1,2,3-tribromopropyl group, iodomethyl group, 1-iodoethyl group, 2-iodoethyl group, 2-iodoisobutyl group, 1,2-diiodoethyl group, 1,3-diiodoisopropyl group, 2,3-diiodo-t-butyl group, 1,2,3-triiodopropyl group, aminomethyl group, 1-aminoethyl group, 2-aminoethyl group, 2-aminoisobutyl group, 1,2-diaminoethyl group, 1,3-diaminoisopropyl group, 2,3-diamino-t-butyl group, 1,2,3-triaminopropyl group, cyanomethyl group, 1-cyanoethyl group, 2-cyanoethyl group, 2-cyanoisobutyl group, 1,2-dicyanoethyl group, 1,3-dicyanoisopropyl group, 2,3-dicyano-t-butyl group, 1,2,3-tricyanopropyl group, nitromethyl group, 1-nitroethyl group, 2-nitroethyl group, 2-nitroisobutyl group, 1,2-dinitroethyl group, 1,3-dinitroisopropyl group, 2,3-dinitro-t-butyl group, 1,2,3-trinitropropyl group, etc.

Examples of the substituted or unsubstituted aralkyl group having 1 to 50 carbon atoms represented by X include benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, 2-β-naphthylisopropyl group, 1-pyrrolylmethyl group, 2-(1-pyrrolyl)ethyl group, p-methylbenzyl group, m-methylbenzyl group, o-methylbenzyl group, p-chlorobenzyl group, m-chlorobenzyl group, o-chlorobenzyl group, p-bromobenzyl group, m-bromobenzyl group, o-bromobenzyl group, p-iodobenzyl group, m-iodobenzyl group, o-iodobenzyl group, p-hydroxybenzyl group, m-hydroxybenzyl group, o-hydroxybenzyl group, p-aminobenzyl group, m-aminobenzyl group, o-aminobenzyl group, p-nitrobenzyl group, m-nitrobenzyl group, o-nitrobenzyl group, p-cyanobenzyl group, m-cyanobenzyl group, o-cyanobenzyl group, 1-hydroxy-2-phenylisopropyl group, 1-chloro-2-phenylisopropyl group, etc.

The substituted or unsubstituted aryloxyl group having 5 to 50 ring atoms represented by X is a group represented by —OY′. Examples of the group represented by Y′ include phenyl group, 1-naphthyl group, 2-naphthyl group, 1-anthryl group, 2-anthryl group, 9-anthryl group, 1-phenanthryl group, 2-phenanthryl group, 3-phenanthryl group, 4-phenanthryl group, 9-phenanthryl group, 1-naphthacenyl group, 2-naphthacenyl group, 9-naphthacenyl group, 1-pyrenyl group, 2-pyrenyl group, 4-pyrenyl group, 2-biphenylyl group, 3-biphenylyl group, 4-biphenylyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-tolyl group, m-tolyl group, p-tolyl group, p-t-butylphenyl group, p (2-phenylpropyl)phenyl group, 3-methyl-2-naphthyl group, 4-methyl-1-naphthyl group, 4-methyl-1-anthryl group, 4′-methylbiphenylyl group, 4″-t-butyl-p-terphenyl-4-yl group, 2-pyrrolyl group, 3-pyrrolyl group, pyradinyl group, 2-pyridinyl group, 3-pyridinyl group, 4-pyridinyl group, 2-indolyl group, 3-indolyl group, 4-indolyl group, 5-indolyl group, 6-indolyl group, 7-indolyl group, 1-isoindolyl group, 3-isoindolyl group, 4-isoindolyl group, 5-isoindolyl group, 6-isoindolyl group, 7-isoindolyl group, 2-furyl group, 3-furyl group, 2-benzofuranyl group, 3-benzofuranyl group, 4-benzofuranyl group, 5-benzofuranyl group, 6-benzofuranyl group, 7-benzofuranyl group, 1-isobenzofuranyl group, 3-isobenzofuranyl group, 4-isobenzofuranyl group, 5-isobenzofuranyl group, 6-isobenzofuranyl group, 7-isobenzofuranyl group, 2-quinolyl group, 3-quinolyl group, 4-quinolyl group, 5-quinolyl group, 6-quinolyl group, 7-quinolyl group, 8-quinolyl group, 1-isoquinolyl group, 3-isoquinolyl group, 4-isoquinolyl group, 5-isoquinolyl group, 6-isoquinolyl group, 7-isoquinolyl group, 8-isoquinolyl group, 2-quinoxanyl group, 5-quinoxanyl group, 6-quinoxanyl group, 1-carbazolyl group, 2-carbazolyl group, 3-carbazolyl group, 4-carbazolyl group, 1-phenanthridinyl group, 2-phenanthridinyl group, 3-phenanthridinyl group, 4-phenanthridinyl group, 6-phenanthridinyl group, 7-phenanthridinyl group, 8-phenanthridinyl group, 9-phenanthridinyl group, 10-phenanthridinyl group, 1-acridinyl group, 2-acridinyl group, 3-acridinyl group, 4-acridinyl group, 9-acridinyl group, 1,7-phenanthrolin-2-yl group, 1,7-phenanthrolin-3-yl group, 1,7-phenanthrolin-4-yl group, 1,7-phenanthrolin-5-yl group, 1,7-phenanthrolin-6-yl group, 1,7-phenanthrolin-8-yl group, 1,7-phenanthrolin-9-yl group, 1,7-phenanthrolin-10-yl group, 1,8-phenanthrolin-2-yl group, 1,8-phenanthrolin-3-yl group, 1,8-phenanthrolin-4-yl group, 1,8-phenanthrolin-5-yl group, 1,8-phenanthrolin-6-yl group, 1,8-phenanthrolin-7-yl group, 1,8-phenanthrolin-9-yl group, 1,8-phenanthrolin-10-yl group, 1,9-phenanthrolin-2-yl group, 1,9-phenanthrolin-3-yl group, 1,9-phenanthrolin-4-yl group, 1,9-phenanthrolin-5-yl group, 1,9-phenanthrolin-6-yl group, 1,9-phenanthrolin-7-yl group, 1,9-phenanthrolin-8-yl group, 1,9-phenanthrolin-10-yl group, 1,10-phenanthrolin-2-yl group, 1,10-phenanthrolin-3-yl group, 1,10-phenanthrolin-4-yl group, 1,10-phenanthrolin-5-yl group, 2,9-phenanthrolin-1-yl group, 2,9-phenanthrolin-3-yl group, 2,9-phenanthrolin-4-yl group, 2,9-phenanthrolin-5-yl group, 2,9-phenanthrolin-6-yl group, 2,9-phenanthrolin-7-yl group, 2,9-phenanthrolin-8-yl group, 2,9-phenanthrolin-10-yl group, 2,8-phenanthrolin-1-yl group, 2,8-phenanthrolin-3-yl group, 2,8-phenanthrolin-4-yl group, 2,8-phenanthrolin-5-yl group, 2,8-phenanthrolin-6-yl group, 2,8-phenanthrolin-7-yl group, 2,8-phenanthrolin-9-yl group, 2,8-phenanthrolin-10-yl group, 2,7-phenanthrolin-1-yl group, 2,7-phenanthrolin-3-yl group, 2,7-phenanthrolin-4-yl group, 2,7-phenanthrolin-5-yl group, 2,7-phenanthrolin-6-yl group, 2,7-phenanthrolin-8-yl group, 2,7-phenanthrolin-9-yl group, 2,7-phenanthrolin-10-yl group, 1-phenazinyl group, 2-phenazinyl group, 1-phenothiazinyl group, 2-phenothiazinyl group, 3-phenothiazinyl group, 4-phenothiazinyl group, 1-phenoxazinyl group, 2-phenoxazinyl group, 3-phenoxazinyl group, 4-phenoxazinyl group, 2-oxazolyl group, 4-oxazolyl group, 5-oxazolyl group, 2-oxadiazolyl group, 5-oxadiazolyl group, 3-furazanyl group, 2-thienyl group, 3-thienyl group, 2-methylpyrrol-1-yl group, 2-methylpyrrol-3-yl group, 2-methylpyrrol-4-yl group, 2-methylpyrrol-5-yl group, 3-methylpyrrol-1-yl group, 3-methyl-pyrrol-2-yl group, 3-methylpyrrol-4-yl group, 3-methylpyrrol-5-yl group, 2-t-butylpyrrol-4-yl group, 3-(2-phenylpropyl)pyrrol-1-yl group, 2-methyl-1-indolyl group, 4-methyl-1-indolyl group, 2-methyl-3-indolyl group, 4-methyl-3-indolyl group, 2-t-butyl-1-indolyl group, 4-t-butyl-1-indolyl group, 2-t-butyl-3-indolyl group, 4-t-butyl-3-indolyl group, etc.

The substituted or unsubstituted arylthio group having 5 to 50 ring atoms represented by X is a group represented by —SY″. Examples of the group represented by Y″ include phenyl group, 1-naphthyl group, 2-naphthyl group, 1-anthryl group, 2-anthryl group, 9-anthryl group, 1-phenanthryl group, 2-phenanthryl group, 3-phenanthryl group, 4-phenanthryl group, 9-phenanthryl group, 1-naphthacenyl group, 2-naphthacenyl group, 9-naphthacenyl group, 1-pyrenyl group, 2-pyrenyl group, 4-pyrenyl group, 2-biphenylyl group, 3-biphenylyl group, 4-biphenylyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-tolyl group, m-tolyl group, p-tolyl group, p-t-butylphenyl group, p-(2-phenylpropyl)phenyl group, 3-methyl-2-naphthyl group, 4-methyl-1-naphthyl group, 4-methyl-1-anthryl group, 4′-methylbiphenylyl group, 4″-t-butyl-p-terphenyl-4-yl group, 2-pyrrolyl group, 3-pyrrolyl group, pyradinyl group, 2-pyridinyl group, 3-pyridinyl group, 4-pyridinyl group, 2-indolyl group, 3-indolyl group, 4-indolyl group, 5-indolyl group, 6-indolyl group, 7-indolyl group, 1-isoindolyl group, 3-isoindolyl group, 4-isoindolyl group, 5-isoindolyl group, 6-isoindolyl group, 7-isoindolyl group, 2-furyl group, 3-furyl group, 2-benzofuranyl group, 3-benzofuranyl group, 4-benzofuranyl group, 5-benzofuranyl group, 6-benzofuranyl group, 7-benzofuranyl group, 1-isobenzofuranyl group, 3-isobenzofuranyl group, 4-isobenzofuranyl group, 5-isobenzofuranyl group, 6-isobenzofuranyl group, 7-isobenzofuranyl group, 2-quinolyl group, 3-quinolyl group, 4-quinolyl group, 5-quinolyl group, 6-quinolyl group, 7-quinolyl group, 8-quinolyl group, 1-isoquinolyl group, 3-isoquinolyl group, 4-isoquinolyl group, 5-isoquinolyl group, 6-isoquinolyl group, 7-isoquinolyl group, 8-isoquinolyl group, 2-quinoxanyl group, 5-quinoxanyl group, 6-quinoxanyl group, 1-carbazolyl group, 2-carbazolyl group, 3-carbazolyl group, 4-carbazolyl group, 1-phenanthridinyl group, 2-phenanthridinyl group, 3-phenanthridinyl group, 4-phenanthridinyl group, 6-phenanthridinyl group, 7-phenanthridinyl group, 8-phenanthridinyl group, 9-phenanthridinyl group, 10-phenanthridinyl group, 1-acridinyl group, 2-acridinyl group, 3-acridinyl group, 4-acridinyl group, 9-acridinyl group, 1,7-phenanthrolin-2-yl group, 1,7-phenanthrolin-3-yl group, 1,7-phenanthrolin-4-yl group, 1,7-phenanthrolin-5-yl group, 1,7-phenanthrolin-6-yl group, 1,7-phenanthrolin-8-yl group, 1,7-phenanthrolin-9-yl group, 1,7-phenanthrolin-10-yl group, 1,8-phenanthrolin-2-yl group, 1,8-phenanthrolin-3-yl group, 1,8-phenanthrolin-4-yl group, 1,8-phenanthrolin-5-yl group, 1,8-phenanthrolin-6-yl group, 1,8-phenanthrolin-7-yl group, 1,8-phenanthrolin-9-yl group, 1,8-phenanthrolin-10-yl group, 1,9-phenanthrolin-2-yl group, 1,9-phenanthrolin-3-yl group, 1,9-phenanthrolin-4-yl group, 1,9-phenanthrolin-5-yl group, 1,9-phenanthrolin-6-yl group, 1,9-phenanthrolin-7-yl group, 1,9-phenanthrolin-8-yl group, 1,9-phenanthrolin-10-yl group, 1,10-phenanthrolin-2-yl group, 1,10-phenanthrolin-3-yl group, 1,10-phenanthrolin-4-yl group, 1,10-phenanthrolin-5-yl group, 2,9-phenanthrolin-1-yl group, 2,9-phenanthrolin-3-yl group, 2,9-phenanthrolin-4-yl group, 2,9-phenanthrolin-5-yl group, 2,9-phenanthrolin-6-yl group, 2,9-phenanthrolin-7-yl group, 2,9-phenanthrolin-8-yl group, 2,9-phenanthrolin-10-yl group, 2,8-phenanthrolin-1-yl group, 2,8-phenanthrolin-3-yl group, 2,8-phenanthrolin-4-yl group, 2,8-phenanthrolin-5-yl group, 2,8-phenanthrolin-6-yl group, 2,8-phenanthrolin-7-yl group, 2,8-phenanthrolin-9-yl group, 2,8-phenanthrolin-10-yl group, 2,7-phenanthrolin-1-yl group, 2,7-phenanthrolin-3-yl group, 2,7-phenanthrolin-4-yl group, 2,7-phenanthrolin-5-yl group, 2,7-phenanthrolin-6-yl group, 2,7-phenanthrolin-8-yl group, 2,7-phenanthrolin-9-yl group, 2,7-phenanthrolin-10-yl group, 1-phenazinyl group, 2-phenazinyl group, 1-phenothiazinyl group, 2-phenothiazinyl group, 3-phenothiazinyl group, 4-phenothiazinyl group, 1-phenoxazinyl group, 2-phenoxazinyl group, 3-phenoxazinyl group, 4-phenoxazinyl group, 2-oxazolyl group, 4-oxazolyl group, 5-oxazolyl group, 2-oxadiazolyl group, 5-oxadiazolyl group, 3-furazanyl group, 2-thienyl group, 3-thienyl group, 2-methylpyrrol-1-yl group, 2-methylpyrrol-3-yl group, 2-methylpyrrol-4-yl group, 2-methylpyrrol-5-yl group, 3-methylpyrrol-1-yl group, 3-methylpyrrol-2-yl group, 3-methylpyrrol-4-yl group, 3-methylpyrrol-5-yl group, 2-t-butylpyrrol-4-yl group, 3-(2-phenylpropyl)pyrrol-1-yl group, 2-methyl-1-indolyl group, 4-methyl-1-indolyl group, 2-methyl-3-indolyl group, 4-methyl-3-indolyl group, 2-t-butyl-1-indolyl group, 4-t-butyl-1-indolyl group, 2-t-butyl-3-indolyl group, 4-t-butyl-3-indolyl group, etc.

The substituted or unsubstituted carboxyl group having 1 to 50 carbon atoms represented by X is a group represented by —COOZ. Examples of Z include methyl group, ethyl group, propyl group, isopropyl group, n-butyl group, s-butyl group, isobutyl group, t-butyl group, n-pentyl group, n-hexyl group, n-heptyl group, n-octyl group, hydroxymethyl group, 1-hydroxyethyl group, 2-hydroxyethyl group, 2-hydroxyisobutyl group, 1,2-dihydroxyethyl group, 1,3-dihydroxyisopropyl group, 2,3-dihydroxy-t-butyl group, 1,2,3-trihydroxypropyl group, chloromethyl group, 1-chloroethyl group, 2-chloroethyl group, 2-chloroisobutyl group, 1,2-dichloroethyl group, 1,3-dichloro isopropyl group, 2,3-dichloro-t-butyl group, 1,2,3-trichloropropyl group, bromomethyl group, 1-bromoethyl group, 2-bromoethyl group, 2-bromo isobutyl group, 1,2-dibromo ethyl group, 1,3-dibromo isopropyl group, 2,3-dibromo-t-butyl group, 1,2,3-tribromopropyl group, iodo methyl group, 1-iodo ethyl group, 2-iodo ethyl group, 2-iodo isobutyl group, 1,2-diiodo ethyl group, 1,3-diiodo isopropyl group, 2,3-diiodo-t-butyl group, 1,2,3-triiodopropyl group, aminomethyl group, 1-amino ethyl group, 2-amino ethyl group, 2-amino isobutyl group, 1,2-diamino ethyl group, 1,3-diamino isopropyl group, 2,3-diamino-t-butyl group, 1,2,3-triaminopropyl group, cyanomethyl group, 1-cyanoethyl group, 2-cyanoethyl group, 2-cyano isobutyl group, 1,2-dicyano ethyl group, 1,3-dicyano isopropyl group, 2,3-dicyano-t-butyl group, 1,2,3-tricyanopropyl group, nitromethyl group, 1-nitroethyl group, 2-nitroethyl group, 2-nitro isobutyl group, 1,2-dinitro ethyl group, 1,3-dinitro isopropyl group, 2,3-dinitro-t-butyl group, 1,2,3-trinitropropyl group, etc.

Examples of the substituted or unsubstituted styryl group represented by X include 2-phenyl-1-vinyl group, 2,2-diphenyl-1-vinyl group, 1,2,2-triphenyl-1-vinyl group, etc.

Examples of the halogen group represented by X include fluorine atom, chlorine atom, bromine atom, iodine atom, etc.

In the general formula (1), m represents an integer 1 to 5, n represents an integer of 0 to 6. It is preferable that m is 1 or 2, and that n is 0 to 4. Further, when m≧2, Ar's within parentheses ( ) may be the same with or different from each other. Also, when n≧2, X's within parentheses ( ) may be the same with or different from each other.

Preferable host materials to be used for the light emitting layer include compounds represented by the following general formulae (i) to (ix).

An asymmetric anthracene represented by the following general formula (i): 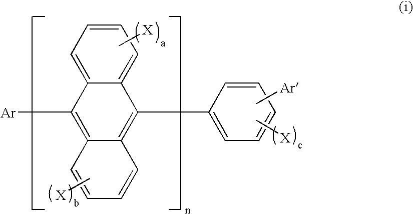
(In the above formula, Ar represents a substituted or unsubstituted fused aromatic group having 10 to 50 ring carbon atoms;

Ar′ represents a substituted or unsubstituted aromatic group having 6 to 50 ring carbon atoms;

X represents a substituted or unsubstituted aromatic group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring atoms, a substituted or unsubstituted arylthio group having 5 to 50 ring atoms, a substituted or unsubstituted alkoxycarbonyl group having 1 to 50 carbon atoms, a carboxyl group, a halogen atom, a cyano group, a nitro group or a hydroxyl group.

a, b and c each independently represents an integer of 0 to 4;

n represents an integer of 1 to 3, with the proviso that when n is an integer of 2 or greater, the plural groups within square brackets [ ] may be the same with or different from each other.)

An asymmetric monoanthracene derivative represented by the following general formula (ii): 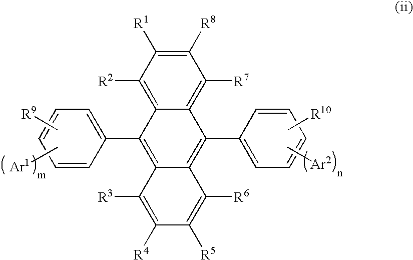
(In the formula, Ar¹ and Ar² each independently represents a substituted or unsubstituted aromatic ring group having 6 to 50 ring carbon atoms; m and n each represents an integer of 1 to 4; with the proviso that in a case where m=n=1 and each bonding position of Ar¹ and Ar² to a benzene ring is bilaterally symmetric to each other, Ar¹ is different from Ar², and in a case where m or n represents an integer of 2 to 4, m is different from n;

R¹ to R¹⁰ each independently represents a hydrogen atom, a substituted or unsubstituted aromatic ring group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted cycloalkyl group, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring atoms, a substituted or unsubstituted arylthio group having 5 to 50 ring atoms, a substituted or unsubstituted alkoxycarbonyl group having 1 to 50 carbon atoms, a substituted or unsubstituted silyl group, a carboxyl group, a halogen atom, a cyano group, a nitro group and a hydroxyl group.)

An asymmetric pyrene derivative represented by the following general formula (iii): 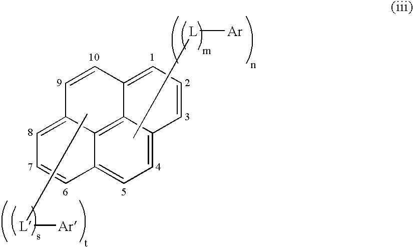
[In the formula, Ar and Ar′ each represents a substituted or unsubstituted aromatic group having 6 to 50 ring carbon atoms;

L and L′ each represents a substituted or unsubstituted phenylene group, a substituted or unsubstituted naphthalenylene group, a substituted or unsubstituted fluorenylene group or a substituted or unsubstituted dibenzosilolylene group;

m represents an integer of 0 to 2, n represents an integer of 1 to 4, s represents an integer of 0 to 2 and t represents an integer of 0 to 4;

L or Ar is bonded to any one of 1- to 5-positions of pyrene ring; and L′ or Ar′ is bonded to any one of 6- to 10-positions of pyrene ring; with the proviso that when n+t represents an even number, Ar, Ar′, L and L′ satisfy the following conditions (1) or (2):

(1) Ar≠Ar′ and/or L≠L′ (wherein ≠ means that each group has a different structure)

(2) when Ar═Ar′ and L=L′

(2-1) m≠s, and/or n≠t, or

(2-2) when m=s and n=t,
(2-2-1) L and L′, or the pyrene ring, are respectively bonded to different positions of Ar and Ar′, or
(2-2-2) in the case where L and L′, or the pyrene ring, are respectively bonded to the same position of Ar and Ar′, the case where L and L′, or Ar and Ar′ are bonded to 1- and 6-positions or 2- and 7-positions of the pyrene ring is excluded.]

An asymmetric anthracene derivative represented by the following general formula (Iv): 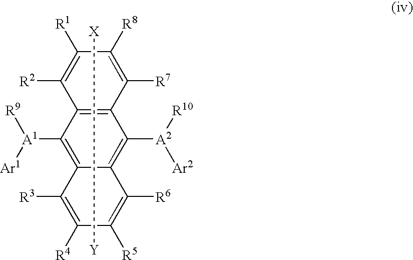
(In the formula, A¹ and A² each independently represents a substituted or unsubstituted fused aromatic ring group having 10 to 20 ring carbon atoms;

Ar¹ and Ar² each independently represents a hydrogen atom, or a substituted or unsubstituted aromatic ring group having 6 to 50 ring carbon atoms,

R¹ to R¹⁰ each independently represents a hydrogen atom, a substituted or unsubstituted aromatic ring group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted cycloalkyl group, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring atoms, a substituted or unsubstituted arylthio group having 5 to 50 ring atoms, a substituted or unsubstituted alkoxycarbonyl group having 1 to 50 carbon atoms, a substituted or unsubstituted silyl group, a carboxyl group, a halogen atom, a cyano group, a nitro group and a hydroxyl group.

Ar¹, Ar², R⁹ and R¹⁰ each may be more than one, and two neighboring groups thereof may form a saturated or unsaturated ring structure, with the proviso that the groups at 9- and 10-positions of the central anthracene in the general formula (1) are not symmetrical with respect to the X-Y axis.

As the material employed for the light emitting layer, the following anthracene derivatives are further preferable:
A1-L-A2 (II)
(In the formula, A1 and A2 each independently represents a substituted or unsubstituted monophenylanthryl group or a substituted or unsubstituted diphenylanthryl group, which may be the same as or different from each other, and L represents a single bond or a divalent bonding group.)

Anthracene derivative represented by the following general formula (III):
A3-An-A4 (III)
(In the formula, An represents a substituted or unsubstituted divalent anthracene residue, A3 and A4 each independently represents monovalent fused aromatic ring group or a substituted or unsubstituted non-fused ring-based aryl group having 12 or more carbon atoms, which may be the same with or different from each other.)

Examples of the anthracene derivative represented by the general formula (II) include the anthracene derivative represented by the general formula (II-a): 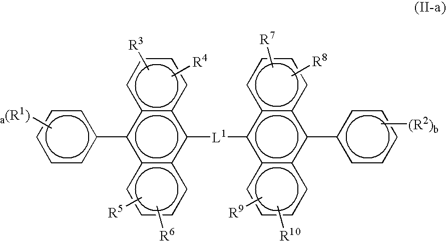
(In the formula, R¹ to R¹⁰ each independently represents a hydrogen atom, an alkyl group, a cycloalkyl group, an aryl group which may be substituted, an alkoxyl group, an aryloxy group, an alkylamino group, an alkenyl group, an arylamino group or a heterocyclic group which may be substituted; a and b each represents an integer of 1 to 5, and when each of a and b is 2 or greater, R¹'s or R²'s may be the same with or different from each other, and R¹ 's or R²'s may bond each other to form a ring; each pair of R³ and R⁴, R⁵ and R⁶, R⁷ and R⁸, and R⁹ and R¹⁰ may bond each other to form a ring, L¹ represents a single bond, —O—, —S—, —N(R)—, an alkylene group or an arylene group; wherein R represents an alkyl group, or an aryl group which may be substituted.)

or the following general formula (II-b): 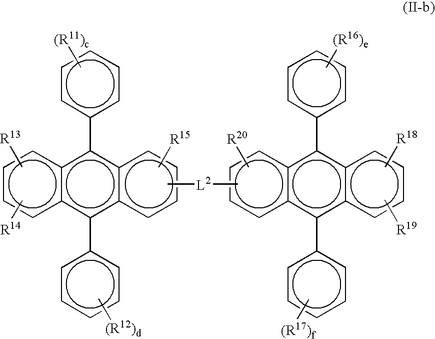
(In the formula, R¹¹ to R²⁰ each independently represents a hydrogen atom, an alkyl group, a cycloalkyl group, an aryl group, an alkoxyl group, an aryloxy group, an alkylamino group, an arylamino group or a heterocyclic group which may be substituted; c, d, e and f each represents an integer of 1 to 5, and when each of c, d, e and f is 2 or greater, R¹¹'s, R¹²'s, R¹⁶'s or R¹⁷'s may be the same with or different from each other, and R¹¹ 's, R¹²'s, R¹⁶'s or R¹⁷'s may bond each other to form a ring; each pair of R¹³ and R¹⁴, and R¹⁸ and R¹⁹ may bond each other to form a ring; L² represents a single bond, —O—, —S—, —N(R)—, an alkylene group or an arylene group wherein R represents an alkyl group or an aryl group which may be substituted.)

Additionally, “which may be substituted” means “substituted or unsubstituted” in the present invention.

In the above general formulae (II-a) and (II-b), the alkyl group among R¹ to R²⁰ preferably has 1 to 6 carbon atoms, the cycloalkyl group preferably has 3 to 6 carbon atoms, the aryl group preferably has 5 to 18 carbon atoms, the alkoxyl group preferably has 1 to 6 carbon atoms, the aryloxy group preferably has 5 to 18 carbon atoms, the arylamino group is preferably an amino group substituted with an aryl group having 5 to 16 carbon atoms; and the preferable examples of the heterocyclic group include triazole group, oxadiazole group, quinoxaline group, furanyl group and thienyl group or so.

Further, it is preferable for the alkyl group represented by R in —N(R)— of L¹ and L² to have 1 to 6 carbon atoms, for the aryl group to have 5 to 18 carbon atoms.

A spirofluorene derivative represented by the following general formula (vii): 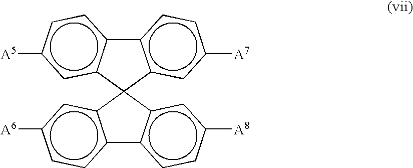
(In the formula, A⁵ to A⁸ each independently represents a substituted or unsubstituted biphenyl group or a substituted or unsubstituted naphthyl group.)

A compound containing a fused ring represented by a following general formula (viii): 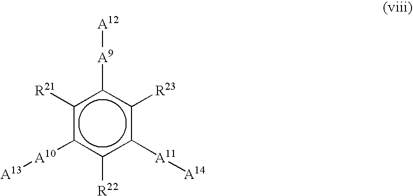
(In the formula, A⁹ to A¹⁴ are same as described above; R²¹ to R²³ each independently represents a hydrogen atom, an alkyl group having 1 to 6 carbon atoms, a cycloalkyl group having 3 to 6 carbon atoms, an alkoxyl group having 1 to 6 carbon atoms, an aryloxy group having 5 to 18 carbon atoms, an aralkyloxy group having 7 to 18 carbon atoms, an arylamino group having 5 to 16 carbon atoms, a nitro group, a cyano group, an ester group having 1 to 6 carbon atoms or a halogen atom; and at least one of A⁹ to A¹⁴ represents a fused aromatic ring having 3 or more rings.)

A fluorene compound represented by the following general formula (ix): 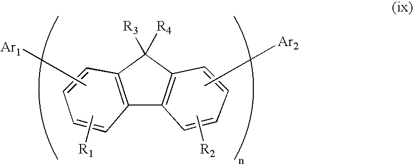
(In the formula, R₁ and R₂ each independently represents a hydrogen atom, a substituted or unsubstituted alkyl group, a substituted or unsubstituted aralkyl group, a substituted or unsubstituted aryl group, a substituted or unsubstituted heterocyclic group, a substituted amino group, a cyano group or a halogen atom; R₁ 's and R₂'s bonding to different fluorene groups may be respectively the same or different, and R₁ and R₂ bonding to the same fluorene group may be the same or different; R₃ and R₄ each independently represents a hydrogen atom, a substituted or unsubstituted alkyl group, a substituted or unsubstituted aralkyl group, a substituted or unsubstituted aryl group, or a substituted or unsubstituted heterocyclic group, and R₃'s and R₄'s bonding to different fluorene groups may be respectively the same or different, and R₃ and R₄ bonding to the same fluorene group may be the same or different; Ar₁ and Ar₂ may be the same or different and each independently represents a substituted or unsubstituted fused polycyclic aromatic group having 3 or more benzene rings or a substituted or unsubstituted fused polycyclic heterocyclic group which has 3 or more benzene rings and hetero rings in total and is bonded to the fluorene group via carbon atom; and n represents an integer of 1 to 10.)

Among the above host materials, an anthracene derivative is preferable and a monoanthracene derivative is more preferable, further an asymmetric anthracene is particularly preferable.

In addition, a phosphorescent compound may be employed as a light emitting material for dopant. A compound containing a carbazole ring for a host material is preferable as the phosphorescent compound. The dopant is not limited as long as it is a a compound capable of emitting light from triplet exciton, and preferably a metal complex containing at least one metal selected from the group consisting of Ir, Ru, Pd, Pt, Os and Re.

A suitable host for phosphorescence composed of a compound containing a carbazole ring is a compound having a function of making the phosphorescent compound to emit light by the energy transfer from its excited state to the phosphorescent compound. The host compound is not limited as long as capable of transferring the exciton energy to the phosphorescent compound and may be appropriately selected according to the purpose. The host compound may have any group such as a heterocycle in addition to the carbazole ring.

Specific examples of the host compound include a carbazole derivative, a triazole derivative, an oxazole derivative, an oxadiazole derivative, an imidazole derivative, a polyarylalkane derivative, a pyrazoline derivative, a pyrazlone derivative, a phenylenediamine derivative, an arylamine derivative, a chalcone derivative substituted by amino group, a styrylanthracene derivative, a fluorenone derivative, a hydrazone derivative, a stilbene derivative, a silazane derivative, an aromatic tertiary amine compound, a styrylamine compound, an aromatic dimethylidene compound, a porphyrin-based compound, an anthraquinodimethane derivative, an anthrone derivative, a diphenylquinone derivative, a thiopyrandioxide derivative, a carbodimide derivative, a fluorenylidene methane derivative, a distyrylpyrazine derivative, heterocyclic tetracarboxylic anhydride such as a naphthaleneperylene, a phthalocyanine derivative, a metal complex of 8-quinolinol derivative, various metal complexes including a metal complex having a ligand of metallophthalocyanine, benzoxazole or benzothiazole, polysilane compound, an electrically conductive polymeric oligomer such as a poly(N-vinylcarbazole) derivative, an aniline copolymer, a thiophene oligomer and a polythiophene, high-molecular compound such as a polythiophene derivative, a polyphenylene derivative, a polyphenylenevinylene derivative and a polyfluorene derivative. The host compound may be used alone or in combination of two or more.

More specific examples include the following:

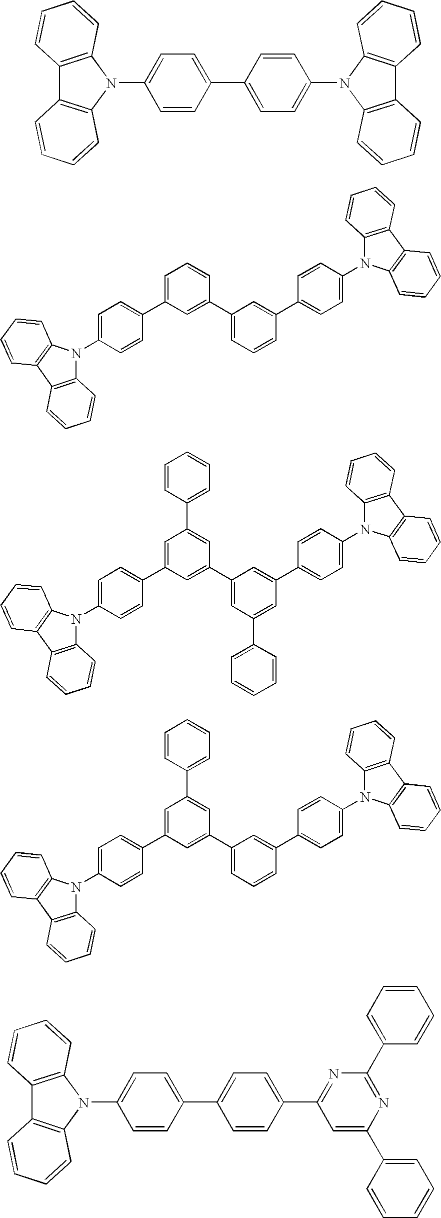

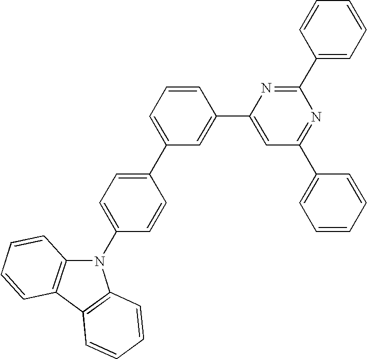

The phosphorescent dopant is a compound capable of emitting light from the triplet exciton. The phosphorescent dopant is not restricted as long as it emits light from the triplet exciton, and preferably a metal complex containing at least one metal selected from the group consisting of Ir, Ru, Pd, Pt, Os and Re, more preferably a porphyrin metal complex or an ortho-metallated metal complex. As the porphyrin metal complex, a porphyrin platinum complex is preferable. The phosphorescent compound may be used alone or in combination of two or more.

There are various ligands to form the ortho-metallated metal complex, and preferred are 2-phenylpyridine derivatives, 7,8-benzoquinoline derivatives, 2-(2-thienyl)pyridine derivatives, 2-(1-naphthyl)pyridine derivatives, and 2-phenylquinoline derivatives. The derivatives may have a substituent as occasion demands. In particular, a dopant introduced with a fluorine atom or a trifluoromethyl group is preferable for the blue light emission. In addition, a ligand such as acetylacetonate and picric acid, other than the above ligands, may be introduced as a co-ligand.

The amount of the phosphorescent dopant in the light emitting layer may be selected for the objective as appropriate without particularly restricted, and for example, it may be selected in the range of from 0.1 to 70% by mass, preferably in the range of from 1 to 30% by mass. The emission is faint and the effect of use is not obtained when the amount is less than 0.1% by mass. The concentration quenching becomes noticeable so that the device performance is deteriorated when the amount exceeds 70% by mass.

With regards to the light emitting layer, it is possible to enhance the light emitting property by adding a slight amount of a fluorescent compound as a dopant. As for the dopant, well-known light emitting materials with a prolonged lifetime can be employed, and a material represented by the following general formula (IV) may be employed as a material for the dopant:

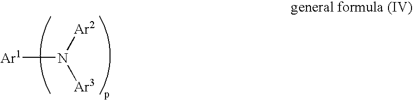

In the formula, Ar¹ to Ar³ each represents a substituted or unsubstituted aromatic group having 6 to 50 ring carbon atoms or a substituted or unsubstituted styryl group.

Examples of the substituted or unsubstituted aromatic group having 6 to 50 ring carbon atoms include phenyl group, 1-naphthyl group, 2-naphthyl group, 1-anthryl group, 2-anthryl group, 9-anthryl group, 1-phenanthryl group, 2-phenanthryl group, 3-phenanthryl group, 4-phenanthryl group, 9-phenanthryl group, 1-naphthacenyl group, 2-naphthacenyl group, 9-naphthacenyl group, 1-pyrenyl group, 2-pyrenyl group, 4-pyrenyl group, 2-biphenylyl group, 3-biphenylyl group, 4-biphenylyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-tolyl group, m-tolyl group, p-tolyl group, p-t-butylphenyl group, p-(2-phenylpropyl)phenyl groups 3-methyl-2-naphthyl group, 4-methyl-1-naphthyl group, 4-methyl-1-anthryl group, 4′-methylbiphenylyl group, 4″-t-butyl-p-terphenyl-4-yl group, 2-fluorenyl group, 9,9-dimethyl-2-fluorenyl group, 3-fluoranthenyl group, etc.

Preferable examples include phenyl group, 1-naphthyl group, 2-naphthyl group, 9-phenanthryl group, 1-naphthacenyl group, 2-naphthacenyl group, 9-naphthacenyl group, 1-pyrenyl group, 2-pyrenyl group, 4-pyrenyl group, 2-biphenylyl group, 3-biphenylyl group, 4-biphenylyl group, o-tolyl group, m-tolyl group, p-tolyl group, p-t-butylphenyl group, 2-fluorenyl group, 9,9-dimethyl-2-fluorenyl group, 3-fluoranthenyl group, etc.

Examples of the substituted or unsubstituted styryl group include 2-phenyl-1-vinyl group, 2,2-diphenyl-1-vinyl group, 1,2,2-triphenyl-1-vinyl group, etc.

p represents an integer of 1 to 4.

Further, when p≧2, Ar²'s and Ar³'s within parentheses ( ) may be the same with or different from each other.

Further, the light emitting layer may contain a hole transporting material, a electron transporting material or a polymer binder, if necessary.

The thickness of the light emitting layer is, in general, selected in the range of from 5 to 50 nm, preferably in the range of from 7 to 50 nm and the most preferably in the range of from 10 to 50 nm.

(V) Hole Injecting and Transporting Layer

The hole injecting and transporting layer is a layer which helps the injection of holes into the light emitting layer and transports the holes to the light emitting region. The layer exhibits a great mobility of holes and, in general, has an ionization energy as small as 5.5 eV or smaller. For the hole injecting and transporting layer, a material which transports holes to the light emitting layer at a small strength of the electric field is preferable. A material which exhibits, for example, a mobility of holes of at least 10⁻⁴ cm²/V·s under an electric field of from 10⁴ to 10⁶ V/cm is preferable.

When the material for the organic EL device of the present invention is employed in the hole transporting region, the hole injecting and transporting layer may be composed of the compound of the present invention alone or in combination with another material.

With regard to the material which may be employed for forming the hole injecting and transporting layer in combination with the material for the organic EL device of the present invention, any material having the foregoing preferable properties is employed without particularly restricted, which is selected from compounds commonly used as a hole transporting material of photoconductive materials and compounds used for forming the hole injecting layer of EL devices. Regarding with the aromatic amine derivative, compounds expressed with the following general formulae are employable.

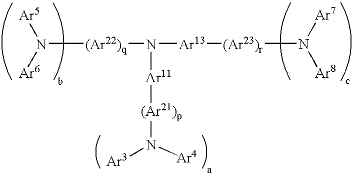

In the above formula, Ar³ to Ar⁸, Ar¹¹ to Ar¹³ and Ar²¹ to Ar²³ each independently represents a substituted or unsubstituted aromatic group having 6 to 50 ring carbon atoms or a substituted or unsubstituted heteroaromatic group having 5 to 50 ring atoms. a to c and p to r each represents an integer of 0 to 3 respectively. Each pair of Ar³ and Ar⁴, Ar⁵ and Ar⁶, and Ar⁷ and Ar⁸ may bond each other to form a saturated or unsaturated ring structure.

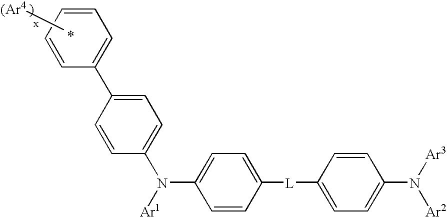

In the above formula, Ar¹ to Ar⁴ each represents a substituted or unsubstituted aromatic group having 6 to 50 ring carbon atoms or a heteroaromatic group having 5 to 50 ring atoms. L is a linking group and represents a single bond, a substituted or unsubstituted aromatic group having 6 to 50 ring carbon atoms or a heteroaromatic group having 5 to 50 ring atoms. X represents an integer of 0 to 5. A pair of Ar² and Ar³ may be bonded each other to form a saturated or unsaturated ring structure.

Specific examples include triazole derivatives (refer to U.S. Pat. No. 3,112,197, etc.), oxadiazole derivatives (refer to U.S. Pat. No. 3,189,447, etc.), imidazole derivatives (refer to JP-B 37-16096, etc.), polyarylalkane derivatives (refer to U.S. Pat. Nos. 3,615,402; 3,820,989; 3,542,544, JP-B 45-555, JP-B 51-10983, JP 51-93224A, JP 55-17105A, JP 56-4148A, JP 55-108667A, JP 55-156953A, JP 56-36656A, etc.), pyrazoline derivatives and pyrazolone derivatives (refer to U.S. Pat. Nos. 3,180,729; 4,278,746; JP 55-88064A, JP 55-88065A, JP 49-105537A, JP 55-51086A, JP 56-80051A, JP 56-88141A, JP 57-45545A, JP 54-112637A, JP 55-74546A, etc.), phenylenediamine derivatives (refer to U.S. Pat. No. 3,615,404; JP-B 51-10105, JP-B 46-3712, JP-B 47-25336, JP 54-119925A, etc.), arylamine derivatives (refer to U.S. Pat. Nos. 3,567,450; 3,240,597; 3,658,520; 4,232,103; 4,175,961; 4,012,376; JP-B 49-35702, JP-B 39-27577, JP 55-144250A, JP 56-119132A, JP 56-22437A, German Patent No. 1,110,518, etc.), amino-substituted chalcone derivatives (refer to U.S. Pat. No. 3,526,501, etc.), oxazole derivatives (disclosed in U.S. Pat. No. 3,257,203, etc.), styrylanthracene derivatives (refer to JP 56-46234A, etc.), fluorenone derivatives (refer to JP 54-110837A, etc.), hydrazone derivatives (refer to U.S. Pat. No. 3,717,462, JP 54-59143A, JP 55-52063A, JP 55-52064A, JP 55-46760A, JP 57-11350A, JP 57-148749A, JP 2-311591A, etc.), stilbene derivatives (refer to JP 61-210363A, JP 61-228451A, JP 61-14642A, JP 61-72255A, JP 62-47646A, JP 62-36674A, JP 62-10652A, JP 62-30255A, JP 60-93455A, JP 60-94462A, JP 60-174749A, JP 60-175052A, etc.), silazane derivatives (U.S. Pat. No. 4,950,950), polysilane-based polymer (JP 2-204996A), aniline-based copolymer (JP 2-282263A), etc.

With regard to the material for the hole injecting and transporting layer, the above materials are also employable, and porphyrin compounds (disclosed in JP 63-295665A, etc.), aromatic tertiary amine compounds and styryl amine compounds (refer to U.S. Pat. No. 4,127,412, JP 53-27033A, JP 54-58445A, JP 55-79450A, JP 55-144250A, JP 56-119132A, JP 61-295558A, JP 61-98353A, JP 63-295695A, etc.) are preferable and the aromatic tertiary amine compounds are particularly preferable.

Further examples include, for example, 4,4′-bis(N-(1-naphthyl)-N-phenylamino)biphenyl (abbreviated as NPD hereinafter) having two fused aromatic rings in its molecule described in U.S. Pat. No. 5,061,569; 4,4′,4″-tris(N-(3-methylphenyl)-N-phenylamino)triphenylamine (abbreviated as MTDATA hereinafter) described in JP 4-308688A made by connecting three triphenylamine units to form a star burst type, etc.

Besides, a compound with heterocyclic derivative having a nitrogen atom expressed with a following general formula disclosed in Japanese Registered Patent No. 03571977 is also employable.

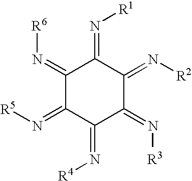

In the formula, R¹, R², R³, R⁴, R⁵ and R⁶ each independently represents any one of a substituted or unsubstituted alkyl group, a substituted or unsubstituted aryl group, a substituted or unsubstituted aralkyl group or a substituted or unsubstituted heterocyclic group. However, R¹, R², R³, R⁴, R⁵ and R⁶ may be the same with or different from each other. Further, each pair of R¹ and R², R³ and R⁴, and R⁵ and R⁶; or each pair of R¹ and R⁶, R² and R³, and R⁴ and R⁵ may be bonded each other to form a fused ring structure.

Furthermore, a compound of the following general formula described in U.S. Pat. No. 2004-0113547 may be employed.

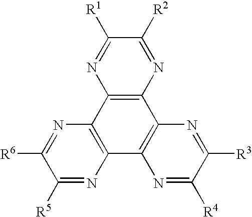

In the formula, R¹ to R⁶ are substituents, and preferably, they each independently represents an electron withdrawing group such as a cyano group, a nitro group, a sulfonyl group, a carbonyl group, a trifluoromethyl group, a halogen atom, etc.

In addition to the above-mentioned aromatic dimethylidene compound described as a material for the light emitting layer, inorganic compound such as p-type Si and p-type SiC may be used as the material for the hole injecting layer.

To form the hole injecting and transporting layer, a thin film may be formed from the above compound in accordance with a well-known process such as the vacuum vapor deposition process, the spin coating process, the casting process and the LB process. Although the thickness of the hole injecting and transporting layer is not particularly limited, the thickness is usually from 5 nm to 5 μm. The hole injecting and transporting layer may be a single layer made of one or more kinds of materials mentioned above or may be laminated with another hole injecting and transporting layer made of a different material, as long as the hole injecting and transporting layer contains the compound of the present invention in its hole transporting region.

An organic semiconductor layer is a layer which assists to inject the holes or to inject the electrons into the light emitting layer, and it is preferable for the organic semiconductor layer to have a conductance of 10⁻¹⁰ S/cm or greater. Examples of the materials for the organic semiconductor layer include electrically conductive oligomers such as an oligomer having thiophene and an oligomer having arylamine disclosed in JP 8-193191A; and electrically conductive dendrimers such as a dendrimer having an arylamine dendrimer.

(VI) Electron Injecting and Transporting Layer

The electron injecting layer is a layer having a great electron mobility, which assists the injection of electrons into the light emitting layer. Among the electron injecting layers, the adhesion improving layer is a layer made of a material exhibiting excellent adhesion to the cathode. As the material for the electron injecting layer, metal complexes of 8-hydroxyquinoline or derivatives thereof are preferable.

Examples of the metal complexes of 8-hydroxyquinoline and derivatives thereof include metal chelate oxinoid compounds including chelates of oxine (in general, 8-quinolinol or 8-hydroxyquinoline).

For example, Alq described in the foregoing term about the light emitting material is employable as the electron injecting layer.

Examples of oxadiazole derivatives include electron transfer compounds represented by the following general formulae:

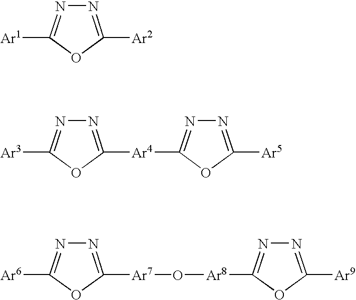

In the formula, Ar¹, Ar², Ar³, Ar⁵, Ar⁶ and Ar⁹ may be the same or different and each independently represents a substituted or unsubstituted aryl group; Ar⁴, Ar⁷ and Ar⁸ each independently represents a substituted or unsubstituted arylene group, which may be the same with or different from each other.

Examples of the aryl group include a phenyl group, a biphenyl group, an anthranyl group, a perilenyl group and a pyrenyl group. Examples of the arylene group include a phenylene group, a naphthylene group, a biphenylene group, an anthranylene group, a perilenylene group, a pyrenylene group, etc. Examples of the substituent include an alkyl group having 1 to 10 carbon atoms, an alkoxy group having 1 to 10 carbon atoms and a cyano group. The electron transfer compound is preferably a thin-film forming compound.

Specific examples of the electron transfer compounds are shown below:

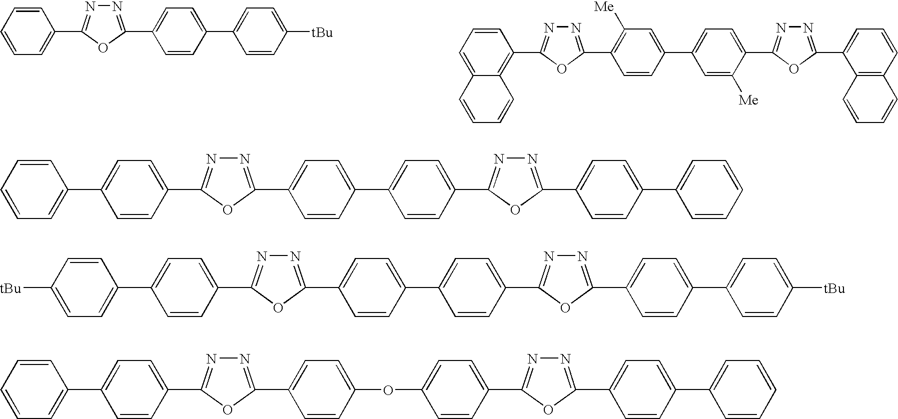

Further, it is known that another compound with heterocycles having a nitrogen atom is preferable as the electron transporting material.

Specific compound preferably employed in the electron injecting layer and the electron transporting layer is a nitrogen-containing heterocyclic derivative represented by the following general formula (4):
HAr-L-Ar¹—Ar² (4)
(In the formula, HAr represents a nitrogen-containing heterocycle having 3 to 40 carbon atoms which may have a substituent; L represents a single bond, an arylene group having 6 to 40 carbon atoms which may have a substituent, or a heteroarylene group having 3 to 40 carbon atoms which may have a substituent; Ar¹ represents a divalent aromatic hydrocarbon group having 6 to 40 carbon atoms which may have a substituent; and Ar² represents an aryl group having 6 to 40 carbon atoms which may have a substituent or a heteroaryl group having 3 to 40 carbon atoms which may have a substituent.)

HAr is selected from the group consisting of:

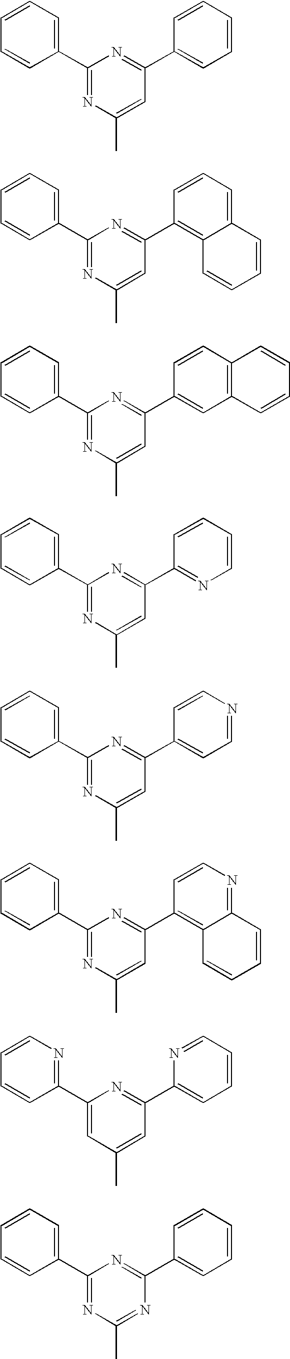

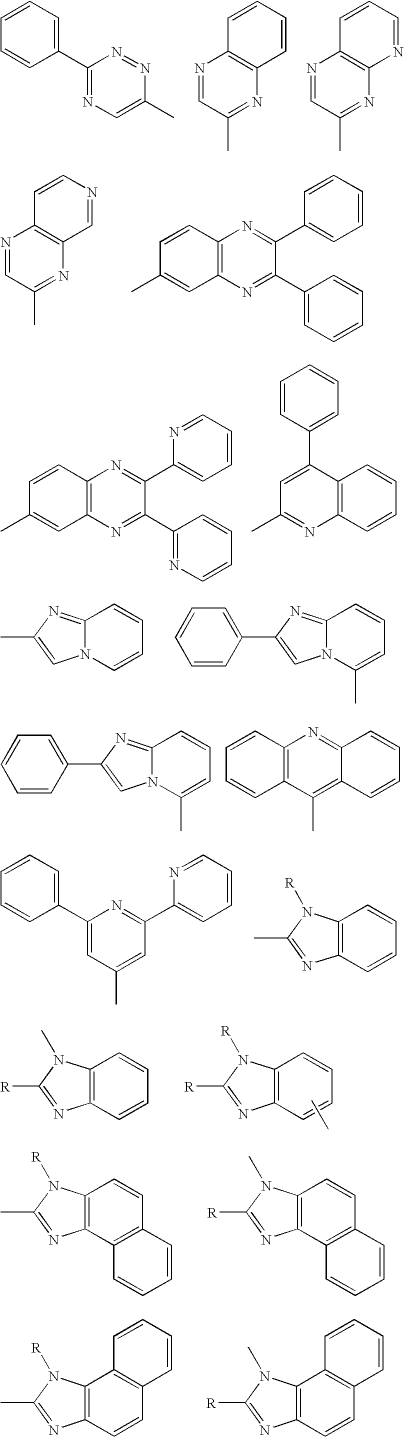

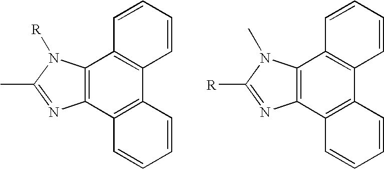

L is selected from the group consisting of:

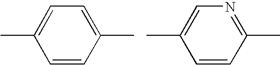

Ar² is selected from the group consisting of: 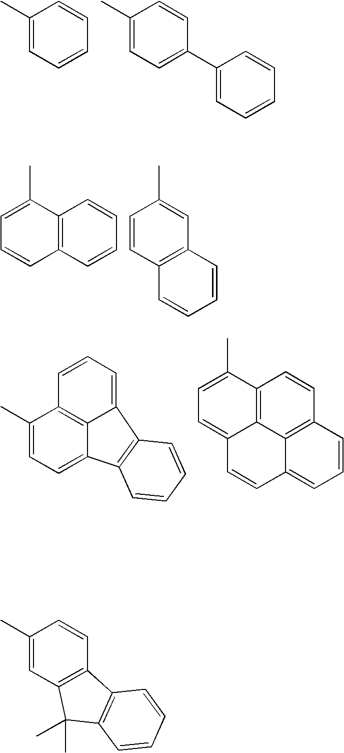
and AR¹ is selected from the following general formula (25) or (26): 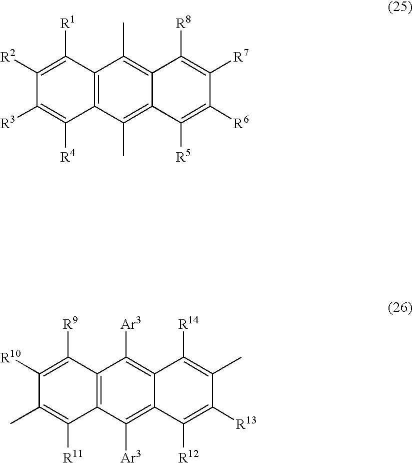
(In the formulae, R¹ to R¹⁴ each independently represents a hydrogen atom, a halogen atom, an alkyl group having 1 to 20 carbon atoms, an alkoxy group having 1 to 20 carbon atoms, an aryloxy group having 6 to 40 carbon atoms, an aryl group having 6 to 40 carbon atoms which may have a substituent or a heteroaryl group having 3 to 40 carbon atoms; and Ar³ represents an aryl group having 6 to 40 carbon atoms which may have a substituent or a heteroaryl group having 3 to 40 carbon atoms.)

Besides, examples include an organic compound satisfying the above constituting condition for the device such as disclosed in JP 9-3448A: 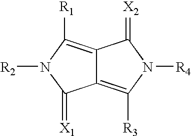
(In the formula, R₁ to R₄ each independently represents a hydrogen atom, a substituted or unsubstituted aliphatic group, a substituted or unsubstituted aliphatic ring group, a substituted or unsubstituted aromatic ring group, a substituted or unsubstituted heterocyclic group; X₁, and X₂ each independently represents an oxygen atom, a sulfur atom or a dicyanomethylene group.) and disclosed in JP 2000-173774A: 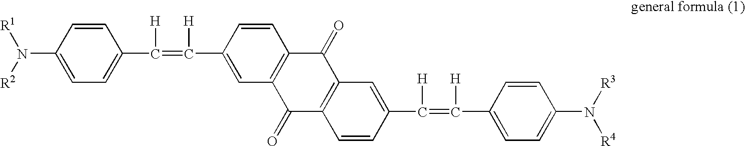
[In the general formula (1), R¹, R², R³ and R⁴ are the same or the different groups and represent an aryl group represented by the following general formula (2): 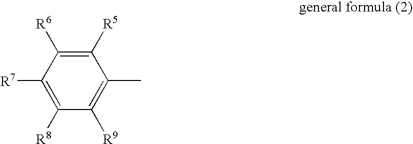
(In the general formula (2), R⁵, R⁶, R⁷, R⁸ and R⁹ are the same or the different groups and represent hydrogen atoms, or alkoxyl groups, alkyl groups, amino groups or alkylamino groups; at least one of which is saturated or unsaturated.)]

Furthermore, any polymer compound including a heterocyclic group having a nitrogen atom or a heterocyclic derivative having a nitrogen atom may be employable.

A preferred embodiment of the device of the present invention contains a reductive dopant in an electron transporting region or an interfacial region between a cathode and an organic compound layer. The reductive dopant is defined as the substance capable of reducing an electron transporting compound. Accordingly, various compounds having a specified reducing property may be employable and preferable examples of the reductive dopant include at least one compound selected from alkali metals, alkaline earth metals, rare earth metals, oxides of alkali metals, halides of alkali metals, oxides of alkaline earth metals, halides of alkaline earth metals, oxides of rare earth metals, halides of rare earth metals, organic complexes of alkali metals, organic complexes of alkaline earth metals, and organic complexes of rare earth metals.

Examples of the preferable reductive dopant include at least one alkali metal selected from a group consisting of Na (the work function: 2.36 eV), K (the work function: 2.28 eV), Rb (the work function: 2.16 eV) and Cs (the work function: 1.95 eV) or at least one alkaline earth metals selected from a group consisting of Ca (the work function: 2.9 eV), Sr (the work function: 2.0 to 2.5 eV) and Ba (the work function: 2.52 eV). A reductive dopant having a work function of 2.9 eV or smaller is particularly preferable. Among those, more preferable reductive dopants include at least one alkali metal selected from the group consisting of K, Rb and Cs, the latter Rb or Cs being further more preferable and the last Cs being the most preferable. Since those alkali metals have a particularly high reducing capability, the luminance is improved and the lifetime is prolonged by the addition thereof into an electron injection region in a relatively small amount. A combination of two or more alkali metals is also preferably used as the reductive dopant having a work function of 2.9 eV or smaller. A combination containing Cs such as Cs and Na, Cs and K, Cs and Rb and Cs, Na and K is particularly preferred. By containing Cs in combination, the reducing capability is effectively performed, and the luminance is enhanced and the lifetime is prolonged in the organic EL device by the addition into the electron injection region.

In the present invention, an electron injecting layer made of an electrically insulating material or a semiconductor may be further disposed between the cathode and the organic layer. The electron injecting layer enables to effectively prevent a leak of electric current and to improve the electron injection property. The electric insulator is preferably at least one metal compound selected from the group consisting of alkali metal chalcogenide, alkaline earth metal chalcogenide, halide of alkali metal and halide of alkaline earth metal. When the electron injecting layer contains these alkali metal chalcogenide or so, it is preferable because the electron injection property is further improved. Preferable examples of the alkali metal chalcogenide include Li₂O, K₂O, Na₂S, Na₂Se and Na₂O. Preferable examples of the alkaline earth metal chalcogenide include CaO, BaO, SrO, BeO, BaS and CaSe. Preferable examples of the alkali metal halide include LiF, NaF, KF, LiCl, KCl and NaCl. Preferable examples of the alkaline earth metal halide include fluorides such as CaF₂, BaF₂, SrF2, MgF₂ and BeF₂ and halides other than the fluorides.

Examples of the semiconductor constituting the electron transporting layer include oxides, nitrides and oxide nitrides containing at least one element selected from Ba, Ca, Sr, Yb, Al, Ga, In, Li, Na, Cd, Mg, Si, Ta, Sb and Zn, which are used singly or in combination of two or more. It is preferable that the inorganic compound for constituting the electron transporting layer is in the form of a crystallite or amorphous insulating thin film. When the electron transporting layer is constituted with the above insulating thin film, a more uniform thin film can be formed and defective pixels such as dark spots can be decreased. Examples of the inorganic compound include the alkali metal chalcogenides, the alkaline earth metal chalcogenides, the alkali metal halides and the alkaline earth metal halides which are described above.

(VII) Cathode

The cathode is formed from an electrode substance such as metal, alloy, electrically conductive compound or a mixture thereof each having a small work function (4 eV or smaller). Examples of the electrode substance include sodium, sodium-potassium alloy, magnesium, lithium, magnesium-silver alloy, aluminum/aluminum oxide, aluminum-lithium alloy, indium, rare earth metal, etc.

The cathode is prepared by forming a thin film of the electrode material described above in accordance with a process such as the vapor deposition process and the sputtering process.

When the light emitted from the light emitting layer is taken out of the cathode, it is preferable that the cathode has a transmittance of greater than 10% to the emitted light.

It is also preferable that the sheet resistivity of the cathode is several hundreds Ω/□ or smaller and the thickness of the cathode is, in general, selected from 10 nm to 1 μm and preferably from 50 to 200 nm.

(VIII) Insulating Layer

In general, an organic EL device tends to form defects in pixels due to leak and short circuit, because an electric field is applied to ultra-thin films. To prevent the defects, a layer of an insulating thin film may be inserted between the pair of electrodes.

Examples of the material for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, vanadium oxide, etc.

Mixtures and laminates of the above compounds can also be employed.

(IX) Fabrication Embodiment of an Organic EL Device

The organic EL device of the present invention is fabricated, for example, by forming an anode, a light emitting layer, an optional hole injecting layer, an optional electron injecting layer, and a cathode in accordance with the process using the materials each being described above. Alternatively, each layer may be formed in a reverse order from the cathode to the anode.

An embodiment of the fabrication of an organic EL device having a construction of anode/hole injecting layer/light emitting layer/electron injecting layer/cathode in this order on a light-transmitting substrate will be described in the following.

First, on a suitable light-transmitting substrate, a thin film of an anode substance is formed so as to have a film thickness of 1 μm or thinner, preferably from 10 nm to 200 nm in accordance with a vapor deposition process, a sputtering process, etc. Then, a hole injecting layer is formed on the anode. The hole injecting layer can be formed in accordance with the vacuum vapor deposition process, the spin coating process, the casting process or the LB process, as described above. The vacuum vapor deposition process is preferable because a uniform film can be easily obtained and pinhole is little formed. When the hole injecting layer is formed in accordance with the vacuum vapor deposition process, the conditions are preferably selected from the following ranges: temperature of deposition source: 50 to 450° C.; degree of vacuum: 10⁻⁷ to 10⁻³ Torr; vapor deposition rate: 0.01 to 50 nm/s; temperature of substrate: −50 to 300° C.; and film thickness: 5 nm to 5 μm; although depending on the employed compound (material for hole injecting layer), the crystal structure and the recombination structure to aim at.

Subsequently, the hole transporting layer is formed on the hole injecting layer by depositing a thin film of the organic light emitting material in accordance with the vacuum vapor deposition process, the sputtering process, the spin coating process or the casting process. The vacuum vapor deposition process is preferable because a uniform film can be easily obtained and pinhole is little formed. When the hole transporting layer is formed in accordance with the vacuum vapor deposition process, the conditions of the vacuum vapor deposition can be selected in the same ranges as in the deposition of the hole injecting layer, although depending on the compound to be used.

Subsequently, the light emitting layer is formed on the hole transporting layer by depositing a thin film of the organic light emitting material in accordance with the vacuum vapor deposition process, the sputtering process, the spin coating process or the casting process. The vacuum vapor deposition process is preferable because a uniform film can be easily obtained and pinhole is little formed. When the light emitting layer is formed in accordance with the vacuum vapor deposition process, the conditions of the vacuum vapor deposition can be selected in the same ranges as in the deposition of the hole transporting layer, although depending on the compound to be used.

Next, the electron transporting layer is formed on the light emitting layer. Similarly to the formation of the hole transporting layer and light emitting layer, the electron transporting layer is preferably formed in accordance with the vacuum vapor deposition process, because a uniform film is required. The conditions of the vacuum vapor deposition can be selected from the same ranges as in the formation of the hole transporting layer and light emitting layer.

Finally, the cathode is formed on the electron injecting layer, to obtain an organic EL device.

The cathode is made of a metal and can be formed in accordance with the vacuum vapor deposition process or the sputtering process. However, the vacuum vapor deposition process is preferably employed in order to prevent the underlying organic layers from being damaged during the formation of the film.

In the above fabrication of the organic EL device, the layers from the anode to the cathode are successively formed preferably after a single evacuation operation.

The process for forming the layers in the organic EL device of the present invention is not particularly limited. A known process such as the vacuum vapor deposition process and the spin coating process or so can be employed. The organic thin film layer containing the compound of the general formula (1) included in the organic EL device of the present invention can be formed in accordance with the vacuum vapor deposition process, the molecular beam epitaxy process (the MBE process) or a known method of coating a solution of the compound in a solvent such as the dipping process, the spin coating process, the casting process, the bar coating process and the roller coating process.

The thickness of each layer in the organic thin film layer in the organic EL device of the present invention is not particularly limited. In general, an excessively thin layer tends to have defects such as pinholes, and an excessively thick layer requires a high-applied voltage and results in decreasing the efficiency. Therefore, the thickness is preferably from several nm to 1 μm.

The organic EL device emits light when a direct voltage of 5 to 40 V is applied with the anode being + terminal and the cathode being − terminal. In the reverse polarity, no electric current flows and no light is emitted upon the application of voltage. When an alternating voltage is applied, the uniform light emission is observed only in the polarity where the anode is + and the cathode is −. The wave shape of alternating voltage is not limited.

Next, the present invention will be described in more detail by reference to the following examples. However, the present invention is not limited to the following examples unless it goes beyond the scope of the gist.

### EXAMPLES

### Synthesis Example 1 Synthesis of Compound 1

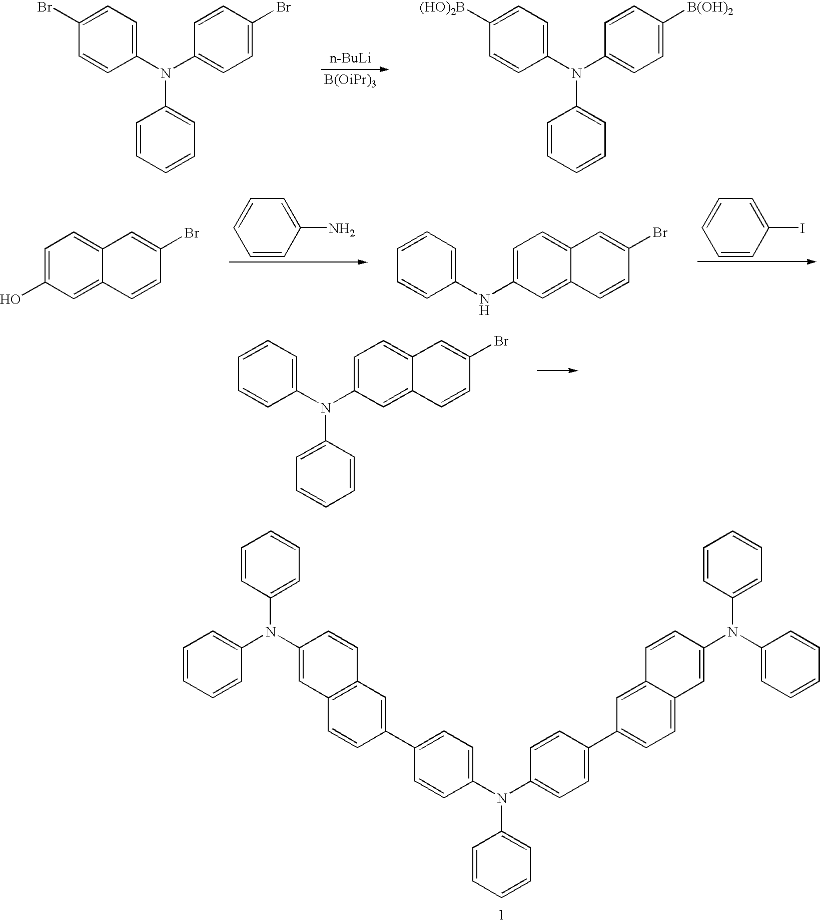

### (1-1) Synthesis of triphenylamine-4,4′-bisboronic acid

Under an atmospheric argon gas, 400-ml dried ethyl ether solution prepared by solving 20.1 g of 4,4′-dibromotriphenylamine was cooled down to −78° C. and 94 ml of 1.6M hexane solution of normal butyllithium was dripped down to the cooled solution. The reaction solution was stirred for 2 h while warming up to 10° C. Cooling the reaction solution down to −78° C. again, a solution prepared by dissolving 56.4 g of triisopropyl borate into 50 ml of dried ether was dripped down to the cooled solution. The reaction solution was stirred at a room temperature for 5 h. Adding 200 ml of 1 N hydrochloric acid and after stirring the resultant solution for 1 h, a water layer was removed. Washing an organic layer with water and saturated aqueous solution of sodium chloride, and after drying over magnesium sulfate, the solvent was removed by distillation under reduced pressure. The resultant solid was purified through silicagel column chromatography to obtain 10.2 g of triphenylamine-4,4′-bisboronic acid.

### (1-2) Synthesis of 2-anilino-6-bromonaphthalene

Under an atmospheric argon gas, 22.3 g of 6-bromo-2-naphthol, 13.9 g of aniline and 0.500 g of iodine were stirred under heating at 200° C. for 8 h. After cooling it down to a room temperature, the reactant was dissolved into dichloromethane. The resultant solution was washed with 200 ml of 10% sodium hydroxide aqueous solution, and dichloromethane was removed by distillation under reduced pressure. After washing with methanol, the resultant solid was purified through silicagel column chromatography to obtain 16.1 g of 2-anilino-6-bromonaphthalene.

### (1-3) Synthesis of 2-bromo-6-(N,N-diphenylamino)naphthalene

Adding 0.881 g of N,N′-dimethylethylenediamine into a solution prepared by mixing 14.9 g of 2-anilino-6-bromonaphthalene, 12.2 g of iodobenzene, 7.2 g of sodium t-butoxide, 0.952 g of copper iodide and 100 ml of xylene, the resultant solution was refluxed with heating under an atmospheric argon gas for 24 h. After cooling the resultant solution down to a room temperature, the solution was filtered and insolubles were removed followed by condensing the filtrate. The residue was purified through silicagel column chromatography to obtain 15.0 g of 2-bromo-6-(N,N-diphenylamino)naphthalene.

### (1-4) Synthesis of Compound 1

Under an argon gas flow, 8.23 g of 2-bromo-6-(N,N-diphenylamino)-naphthalene, 3.33 g of triphenylamine-4,4′-bisboronic acid, 462 mg of tetrakis(triphenylphosphine)palladium(0), 60 ml of toluene and 30 ml of 2M sodium carbonate aqueous solution were prepared as a mixed solution, and the solution was refluxed with heating for 8 h. After the reaction was completed, extraction with toluene was carried out and a water layer was removed. After washing an organic layer with water, dried it over magnesium sulfate. After separating magnesium sulfate by filtration, the organic layer was condensed. The residue was purified through silicagel column chromatography to obtain 5.23 g of pale greenish white solid. As a result of mass spectrum analysis, the pale greenish white solid was identified as the aimed substance, and it was recognized that m/e=831 was attributable to molecular weight of 831.36.

### Synthesis Example 2 Synthesis of Compound 2

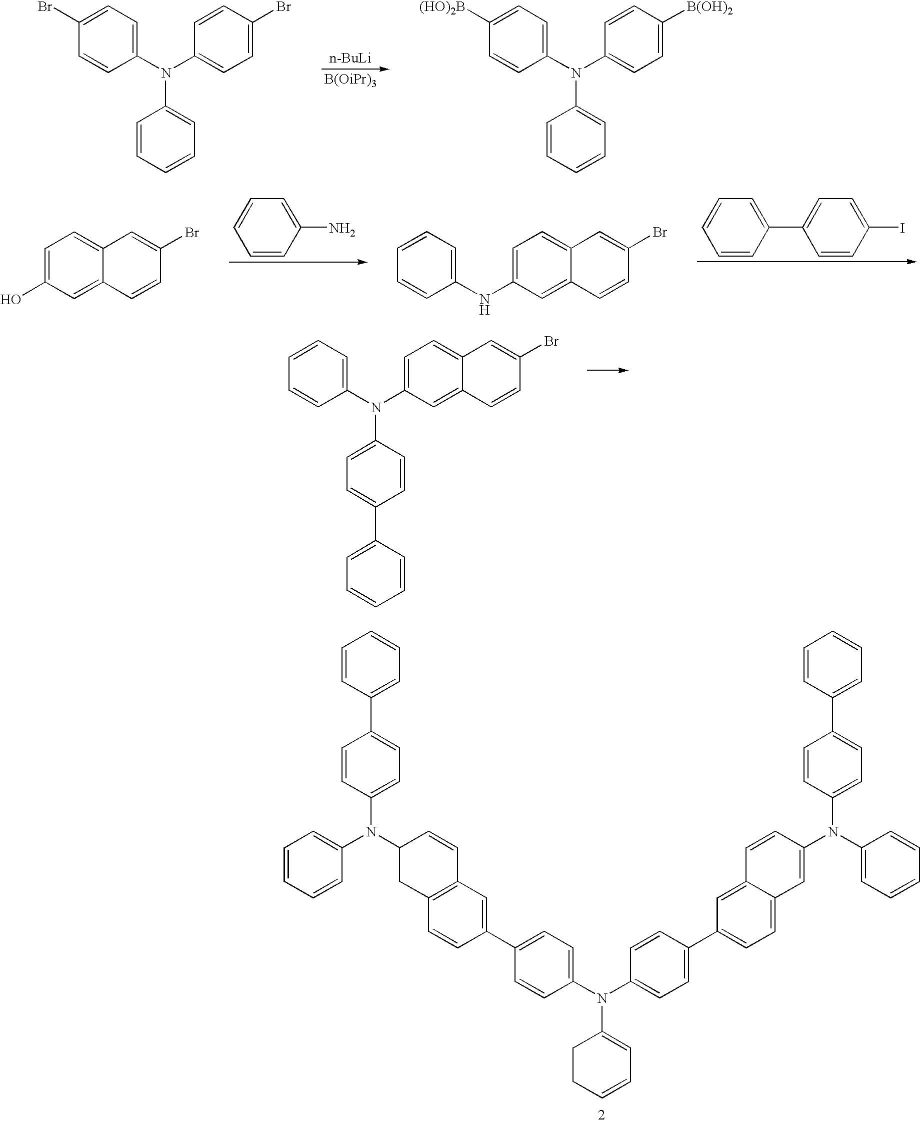

Compound 2 was synthesized in the same manner as the synthesis of Compound 1 except that 4-iodobiphenyl was employed instead of iodobenzene. As a result of mass spectrum analysis, the resultant solid was identified as the aimed substance, and it was recognized that m/e=983 was attributable to molecular weight of 983.42.

### Synthesis Example 3 Synthesis of Compound 3

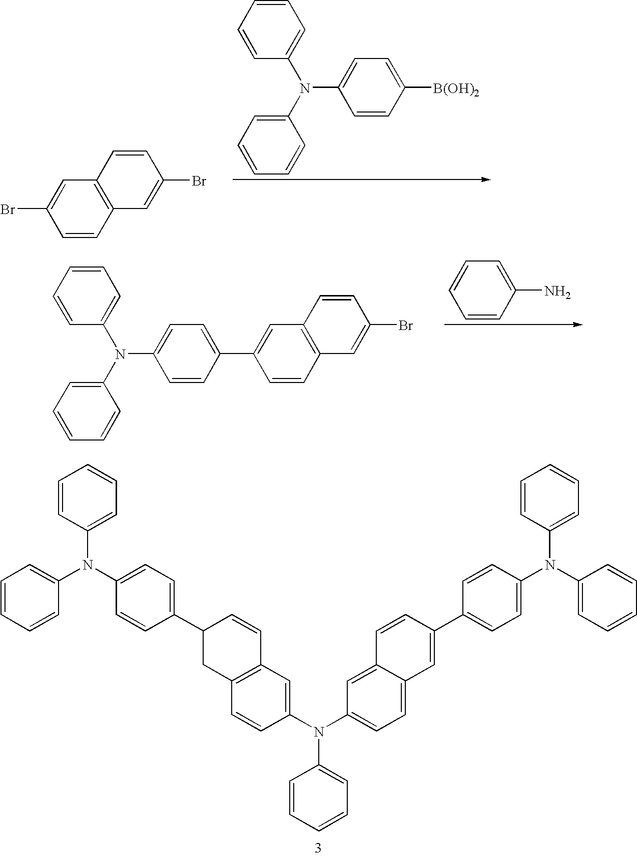

### (3-1) Synthesis of 2-bromo-6-[4-(N,N-diphenylamino)phenyl]naphthalene

Under an argon gas flow, 28.4 g of 2,6-dibromonaphthalene, 28.9 g of triphenylamine-4-boronic acid, 2.31 g of tetrakis(triphenylphosphine)-palladium(0), 300 ml of toluene and 150 ml of 2M sodium carbonate aqueous solution were prepared as a mixed solution, and the solution was refluxed with heating for 8 h. After the reaction was completed, extraction with toluene was carried out and a water layer was removed. After washing an organic layer with water, dried it over magnesium sulfate. After separating magnesium sulfate by filtration, the organic layer was condensed. The residue was purified through silicagel column chromatography to obtain 10.2 g of pale greenish white solid.

### (3-2) Synthesis of Compound 3

Adding 113 μl of 0.66% by weight toluene solution of tri-t-butylphosphine into a mixed solution prepared by dissolving 4.49 g of 2-bromo-6-[4-(N,N-diphenylamino)phenyl]naphthalene, 0.465 g of aniline, 183 mg of tris(dibenzylideneacetone)dipalladium(0) and 1.35 g of sodium t-butoxide into 50 ml solution of toluene, the resultant solution was refluxed with heating for 5 h. After cooling the resultant solution down to a room temperature, the solution was filtered through celite. After the filtrate was condensed, it was purified through silicagel column chromatography to obtain 2.26 g of pale greenish white solid. As a result of mass spectrum analysis, the resultant solid was identified as the aimed substance, and it was recognized that m/e=831 was attributable to molecular weight of 831.36.

### Synthesis Example 4 Synthesis of Compound 4

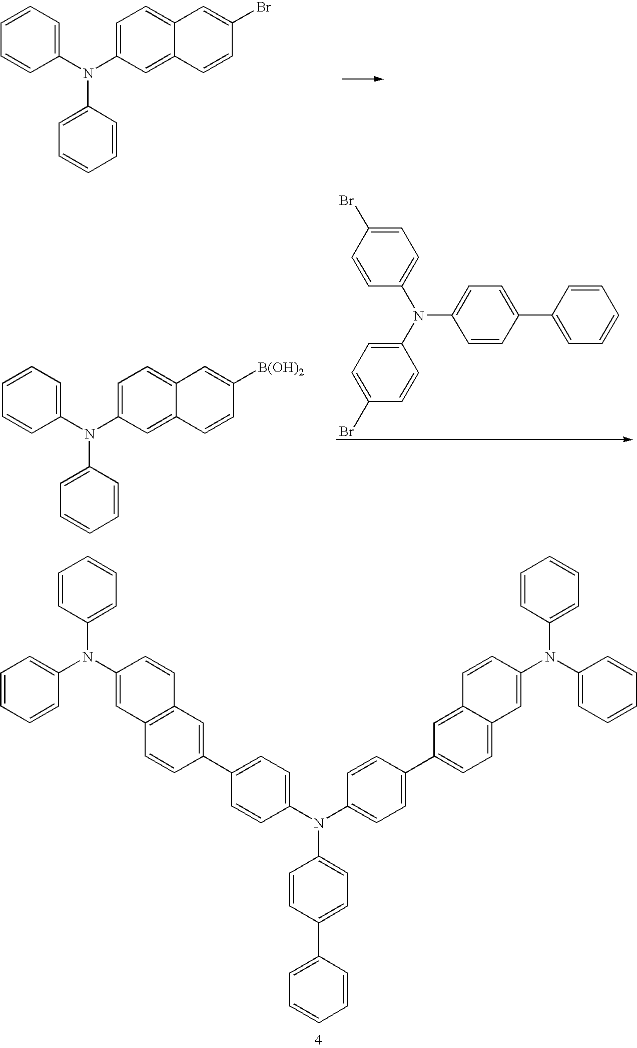

### (4-1) Synthesis of 6-(N,N-diphenylamino)naphthalene-2-boronic acid

Under an atmospheric argon gas, cooling a solution prepared by mixing 37.4 g of 2-bromo-6-(N,N-diphenylamino)naphthalene, 400 ml of dried ethyl ether and 100 ml of dried toluene down to a temperature of −78° C., 65 ml of 1.6M hexane solution of normal butyllithium was dripped into the cooled solution. The reaction solution was stirred for 2 h while warming up to 10° C. Cooling the reaction solution down to −78° C. again, a solution prepared by dissolving 47.0 g of triisopropyl borate into 50 ml of dried ether was dripped down to the cooled solution. The reaction solution was stirred at a room temperature for 5 h. Adding 200 ml of 1 N hydrochloric acid and after stirring the resultant solution for 1 h, a water layer was removed. Washing an organic layer with water and saturated aqueous solution of sodium chloride, and after drying over magnesium sulfate, the solvent was removed by distillation under reduced pressure. The resultant solid was purified through silicagel column chromatography to obtain 25.2 g of 6-(N,N-diphenylamino)naphthalene-2-boronic acid.

### (4-2) Synthesis of Compound 4

Under an argon gas flow, 4.77 g of 4,4′-dibromo-4″-phenyltriphenylamine, 4.07 g of 6-(N,N-diphenylamino)naphthalene-2-boronic acid, 231 mg of tetrakis(triphenylphosphine)palladium(0), 40 ml of toluene and 20 ml of 2M sodium carbonate aqueous solution were prepared as a mixed solution, and the solution was refluxed with heating for 8 h. After the reaction was completed, extraction with toluene was carried out and a water layer was removed. After washing an organic layer with water, dried it over magnesium sulfate. After separating magnesium sulfate by filtration, the organic layer was condensed. The residue was purified through silicagel column chromatography to obtain 4.23 g of pale greenish white solid. As a result of mass spectrum analysis, the resultant solid was identified as the aimed substance, and it was recognized that m/e=907 was attributable to molecular weight of 907.39.

### Synthesis Example 5 Synthesis of Compound 5

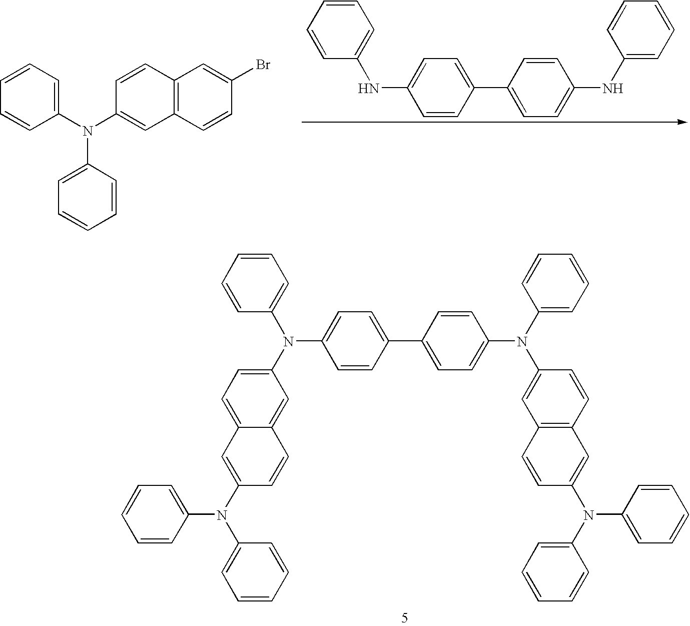

Adding 113 μl of 0.66% by weight toluene solution of tri-t-butylphosphine into a mixed solution prepared by dissolving 8.23 g of 2-bromo-6-[4-(N,N-diphenylamino)phenyl]naphthalene, 3.36 g of N,N′-diphenyl benzidine, 183 mg of tris(dibenzylideneacetone)-dipalladium(0) and a solution of 2.69 g of sodium t-butoxide in 50 ml of toluene, the resultant solution was refluxed with heating for 5 h. After cooling the resultant solution down to a room temperature, the solution was filtered. The insolubles were washed with methanol, water and methanol successively and dried. The resultant solid was re-crystallized from toluene to obtain 6.23 g of pale yellow crystal. As a result of mass spectrum analysis, the pale yellow crystal was identified as the aimed substance, and it was recognized that m/e=922 was attributable to molecular weight of 922.40.

### Example 1

A glass substrate (manufactured by GEOMATEC Company) of 25 mm×75 mm×1.1 mm thickness having an ITO transparent electrode was cleaned by application of ultrasonic wave in isopropyl alcohol for 5 min and then by exposure to ozone generated by ultraviolet light for 30 min. The cleaned glass substrate having the transparent electrode lines was attached to a substrate holder of a vacuum vapor deposition apparatus. On the surface of the cleaned substrate at the side having the transparent electrode, a film of Compound 1 having a thickness of 60 nm was formed in accordance with a resistance heating vapor deposition process so as to cover the transparent electrode. The formed film of Compound 1 worked as the hole injecting layer. Continuing to the film formation of the Compound 1, a layer of HT-1 with a structure below having a thickness of 20 nm was formed over the film of Compound 1. Over the film of HT-1, a film of 9-(2-naphthyl)-10-[4-(1-naphthyl)phenyl]anthracene (abbreviated as AN-1 hereunder) having a thickness of 40 nm was formed. At the same time, the following amine compound D-1 having styryl group as a light emitting molecule was deposited with a weight ratio of D-1:AN-1=2:40. The formed film worked as a light emitting layer. On the film formed above, a film of Alq having a thickness 20 nm was formed. The formed film worked as an electron injecting layer. On the film formed above, LiF (film thickness: 1 nm) was deposited from vapor, and metal Al was deposited over LiF thereby forming a metal cathode, and an organic EL device was fabricated.

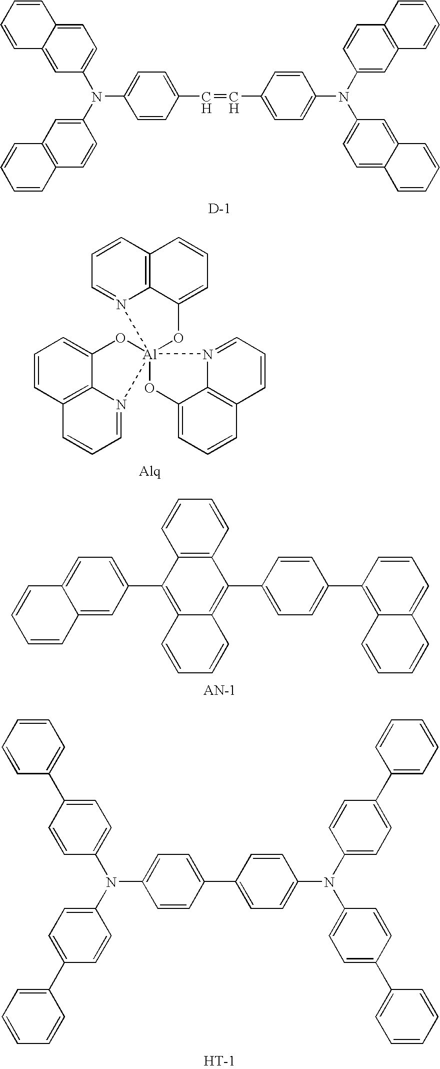

### Example 2

An organic EL device was fabricated in accordance with the same procedures as those conducted in Example 1 except that Compound 2 was employed instead of Compound 1.

### Example 3

An organic EL device was fabricated in accordance with the same procedures as those conducted in Example 1 except that Compound 3 was employed instead of Compound 1.

### Example 4

An organic EL device was fabricated in accordance with the same procedures as those conducted in Example 1 except that Compound 4 was employed instead of Compound 1.

### Example 5

An organic EL device was fabricated in accordance with the same procedures as those conducted in Example 1 except that Compound 5 was employed instead of Compound 1.

### Comparative Example 1

An organic EL device was fabricated in accordance with the same procedures as those conducted in Example 1 except that Compound (A) was employed instead of Compound 1.

### Comparative Example 2

An organic EL device was fabricated in accordance with the same procedures as those conducted in Example 1 except that Compound (B) was employed instead of Compound 1.

### Comparative Example 3

An organic EL device was fabricated in accordance with the same procedures as those conducted in Example 1 except that Compound (C) was employed instead of Compound 1.

### Comparative Example 4

An organic EL device was fabricated in accordance with the same procedures as those conducted in Example 1 except that Compound (D) was employed instead of Compound 1.

### Comparative Example 5

An organic EL device was fabricated in accordance with the same procedures as those conducted in Example 1 except that Compound (E) was employed instead of Compound 1.

### Comparative Example 6

An organic EL device was fabricated in accordance with the same procedures as those conducted in Example 1 except that Compound (F) was employed instead of Compound 1.

### Comparative Example 7

An organic EL device was fabricated in accordance with the same procedures as those conducted in Example 1 except that Compound (G) was employed instead of Compound 1.

### Comparative Example 8

An organic EL device was fabricated in accordance with the same procedures as those conducted in Example 1 except that Compound (H) was employed instead of Compound 1.

### Comparative Example 9

An organic EL device was fabricated in accordance with the same procedures as those conducted in Example 1 except that Compound (I) was employed instead of Compound 1.

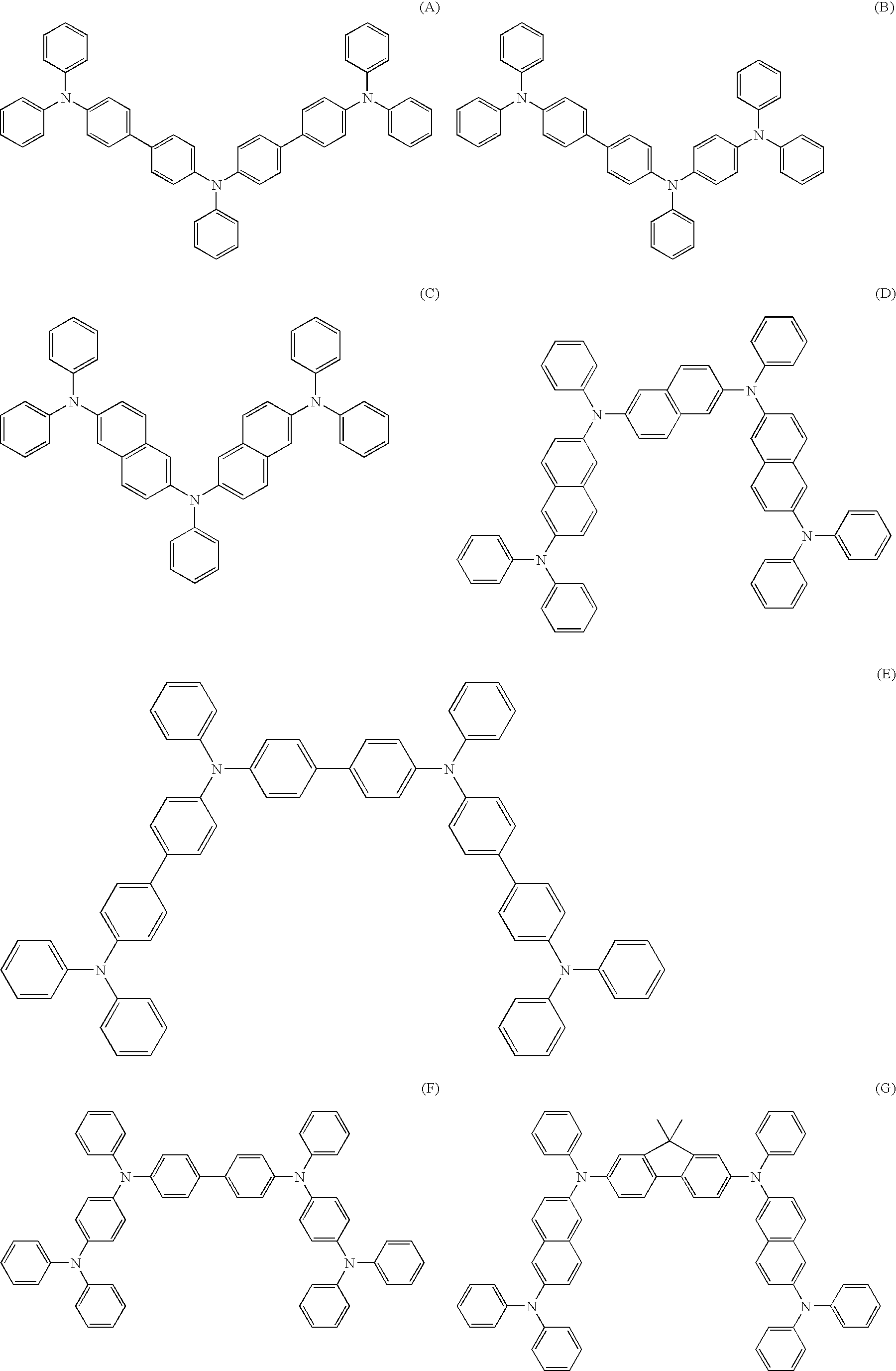

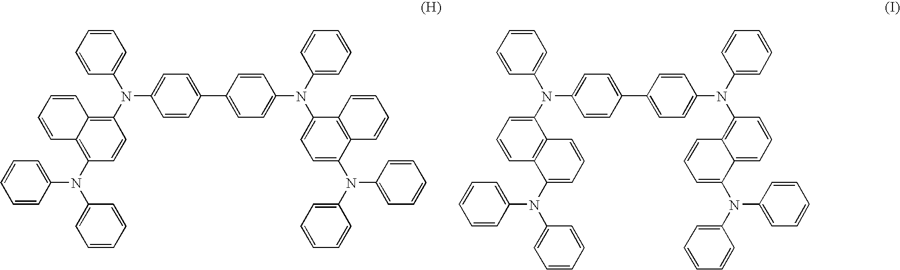

Results of device performance about Examples 1 to 5 and Comparative Examples 1 to 9 are shown in Table 1 below:

TABLE 1
Device performance driven at 10 mA/cm² and half lifetime at initial
luminance of 1000 cd/m²

| | | | | |
|---|---|---|---|---|
| Hole | Electric | Emission | | |
| injecting | voltage | efficiency | Luminescent | |
| material | (V) | (cd/A) | color | Lifetime |

Examples

| | | | | | |
|---|---|---|---|---|---|
| 1 | 1 | 6.4 | 6.5 | Blue | 7000 |
| 2 | 2 | 6.4 | 6.5 | Blue | 7000 |
| 3 | 3 | 6.4 | 6.5 | Blue | 7000 |
| 4 | 4 | 6.4 | 6.5 | Blue | 7000 |
| 5 | 5 | 6.3 | 6.6 | Blue | 8000 |

Comparative Examples

| | | | | | |
|---|---|---|---|---|---|
| 1 | (A) | 6.9 | 5.9 | Blue | 4000 |
| 2 | (B) | 8.4 | 5.9 | Blue | 4000 |
| 3 | (C) | 8.4 | 5.9 | Blue | 3000 |
| 4 | (D) | 7.4 | 5.5 | Blue | 4000 |
| 5 | (E) | 6.9 | 6.2 | Blue | 6000 |
| 6 | (F) | 8.9 | 6.2 | Blue | 6000 |
| 7 | (G) | 7.5 | 5.0 | Blue | 1000 |
| 8 | (H) | 8.5 | 5.5 | Blue | 1000 |
| 9 | (I) | 8.4 | 5.5 | Blue | 1000 |

Comparison between Examples 1 to 4 and Comparative Examples 1 to 3 proves that a compound having phenyl naphthylene as a linker of the present invention exhibits more prolonged lifetime under a low electric voltage than a compound having biphenylene, phenylene or naphthylene as a linker. In other words, an insertion of a linker between naphthalene and amine of a diamino naphthalene skeleton peculiarly reduces a driving voltage.

Further, comparison between Example 5 and Comparative Examples 4 to 9 proves that a compound having naphthylene linker exhibits more prolonged lifetime under a low electric voltage even about tetraamine compounds. Particularly paying attention to Compound (G) and a compound of the present invention, even a difference between fluorene and biphenyl in a central skeleton exhibits that the compound of the present invention prolongs its lifetime drastically. While a fluorene compound reduces its lifetime because it decomposes at vapor deposition, a tetraamine compound of the present invention is capable of being vapor deposited stably, conceivably enabling to prolong its lifetime.

Furthermore, the compound of the present invention is operable under a lower electric voltage than Compound (E) whose linkers are all biphenyls. A peculiar effect of the naphthylene linker that changing a part of Compound (E) to the naphthylene linker improves its flatness and also improves adhesion with an anode conceivably reducing the driving voltage.

As described above, an employment of the compound of the present invention as a hole injecting material provides a low electric voltage operation, an enhanced efficiency and a prolonged lifetime.

### INDUSTRIAL APPLICABILITY

As described in detail above, the organic EL device employing the aromatic amine derivative and the material for the organic EL device of the present invention provides an enhanced efficiency of light emission and a blue light emission of prolonged lifetime. Therefore, the device is extremely useful as a highly practical organic EL device. Therefore, the organic electroluminescence device of the present invention is useful for a planar light emitting member for wall televisions, a light source for a back light of displays and the like.

1: An aromatic amine derivative represented by the following general formula (1): 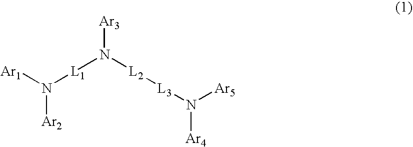
wherein Ar₁ to Ar₅ each independently represents a substituted or unsubstituted aryl group having 6 to 50 ring carbon atoms;
L₁ to L₃ each independently represents a substituted or unsubstituted arylene group having 6 to 50 ring carbon atoms; with the proviso that at least one of L₁ to L₃ represents a substituted or unsubstituted arylene group which comprises a fused aromatic ring having 6 to 50 ring carbon atoms.

2: An aromatic amine derivative represented by the following general formula (2): 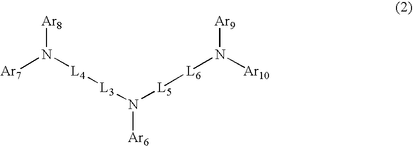
wherein Ar₆ to Ar₁₀ each independently represents a substituted or unsubstituted aryl group having 6 to 50 ring carbon atoms;
L₃ to L₆ each independently represents a substituted or unsubstituted arylene group having 6 to 50 ring carbon atoms; with the proviso that at least one of L₃ to L₆ represents a fused aromatic ring group having 6 to 50 ring carbon atoms.

3: The aromatic amine derivative according to claim 2, wherein at least one of L₃ to L₆ in the general formula (2) is a group comprising a substituted or unsubstituted naphthalene.

4: The aromatic amine derivative according to claim 3, wherein the group comprising the substituted or unsubstituted naphthalene is represented by any of the following general formulae (3) and (4): 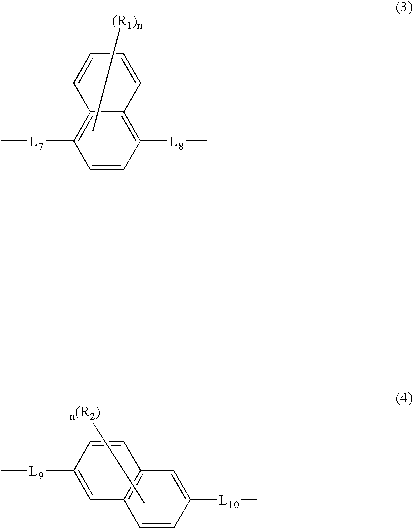
wherein R₁ and R₂ each independently represents a substituent, n represents an integer of 0 to 6; and
L₇ to L₁₀ each independently represents a single bond or a substituted or unsubstituted arylene group having 6 to 50 ring carbon atoms.

5: An aromatic amine derivative represented by the following general formula (5): 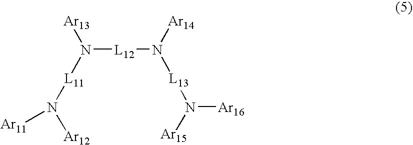
wherein Ar₁₁ to Ar₁₆ each independently represents a substituted or unsubstituted aryl group having 6 to 50 ring carbon atoms;
L₁₁ and L₁₃ each independently represents a single bond or a substituted or unsubstituted arylene group having 6 to 50 ring carbon atoms;
L₁₂ is represented by the following general formula (6): 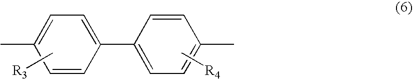
wherein R₃ and R₄ each independently represents a substituent;
at least one of L₁₁ and L₁₃ corresponds to a naphthylene derivative represented by the following general formula (7): 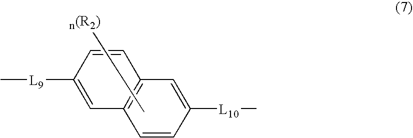
wherein R₂ represents a substituent, n represents an integer of 0 to 6; and
L₉ and L₁₀ each independently represents a single bond or a substituted or unsubstituted arylene group having 6 to 50 ring carbon atoms.

6: The aromatic amine derivative according to claim 5, which is represented by the following general formula (8): 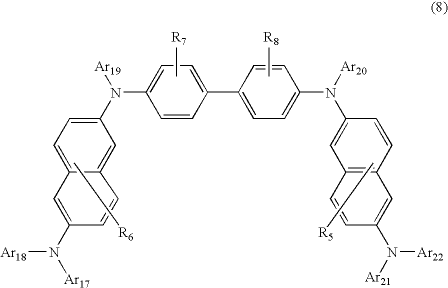
wherein Ar₁₇ to Ar₂₂ each independently represents a substituted or unsubstituted aryl group having 6 to 50 ring carbon atoms; and
R₅ to R₈ represent substituents.

7: The aromatic amine derivative according to any one of claims 1 to 6, wherein the aromatic amine derivative is used as a material for organic electroluminescence devices.

8: The aromatic amine derivative according to any one of claims 1 to 6, wherein the aromatic amine derivative is used as a hole injecting material or a hole transporting material for organic electroluminescence devices.

9: An organic electroluminescence device which comprises one or more organic thin film layers including at least one light emitting layer sandwiched between an anode and a cathode, wherein at least one of the organic thin film layers comprises the aromatic amine derivative according to any one of claims 1 to 6 singly or as its mixture component.

10: The organic electroluminescence device according to claim 9, wherein the organic thin film layers comprise at least one of a hole transporting region and a hole injecting region, and wherein the aromatic amine derivative according to any one of claims 1 to 6 is used in at least one of the hole injecting region and the hole transporting region.

11: The organic electroluminescence device according to claim 9, wherein the organic thin film layers comprise at least one of a hole transporting layer and a hole injecting layer, and wherein the aromatic amine derivative according to any one of claims 1 to 6 is used in at least one of the hole injecting layer and the hole transporting layer.

12: The organic electroluminescence device according to any one of claims 9 to 11, which emits blue light.
